(19) United States
(12) Patent Application Publication
(10) Pub. No.: US 2020/0368239 A1
(43) Pub. Date: Nov. 26, 2020

(54) TREATMENT OF AMYOTROPHIC LATERAL SCLEROSIS USING TRAMETINIB

(71) Applicant: GENUV Inc., Seoul (KR)

(72) Inventors: Sungho HAN, Seoul (KR); Mi-Yeon KIM, Incheon (KR); Sun-Young LEE, Seongnam (KR)

(21) Appl. No.: 16/880,894

(22) Filed: May 21, 2020

Related U.S. Application Data
(60) Provisional application No. 62/851,500, filed on May 22, 2019.

(51) Int. Cl. A61K 31/519; A61P 21/00; C12Q 1/6883; G01N 33/68

(52) U.S. Cl. CPC A61K 31/519; A61P 21/00; C12Q 1/6883; G01N 2800/28; C12Q 2600/106; C12Q 2600/158; G01N 2800/52; G01N 33/68

(57) ABSTRACT

The present invention relates to administration methods and dosage regimens for treatment of neurodegenerative diseases, in particular ALS, using trametinib. The administration methods and dosage regimens induce neural regeneration and changes in gene expression.

### 1. CROSS REFERENCE TO RELATED APPLICATIONS

This application claims the benefit of U.S. Provisional Application No. 62/851,500, filed May 22, 2019, which is hereby incorporated in its entirety by reference.

### 2. BACKGROUND

Amyotrophic lateral sclerosis (“ALS”) is a progressive neurodegenerative disease that affects nerve cells in the brain and the spinal cord. ALS belongs to the group of disorders known as neurodegenerative diseases, and more specifically, to the group of neurodegenerative disorders known as motor neuron disease, which is caused by gradual degeneration and death of motor neurons. When motor neurons degenerate, their ability to initiate and control muscle movement is lost. As activities of voluntary muscles are progressively affected, patients may lose the ability to speak, eat, move and breathe.

Two types of ALS are known—sporadic and familial. Sporadic ALS accounts for 90 percent or more of all ALS cases, and seems to occur at random with no clearly associated risk factors and no family history of the disease. Familial ALS (FALS) accounts for 5 to 10 percent of all ALS cases, which is inherited. Mutations in more than a dozen genes have been found to be associated with familial ALS. About 25 to 40 percent of all familial cases (and a small percentage of sporadic cases) are associated with a defect in a gene known as “chromosome 9 open reading frame 72,” or C9ORF72. Another 12 to 20 percent of familial cases are associated with mutations in the gene that codes for the production of the enzyme copper-zinc superoxide dismutase 1 (SOD1).

Riluzole and edaravone have been approved in the U.S. for the treatment of ALS, but have not yet been shown to extend life of ALS patients by more than a few months. Currently, there is no cure for ALS and no effective treatment to halt, or reverse, the progression of the disease. There is therefore a need for an effective treatment for ALS.

### 3. SUMMARY

Trametinib (SNR1611, Mekinist®) was recently demonstrated to be effective in inducing neuronal differentiation and promoting survival of neurons and neural stem cells (NSCs), as described in U.S. Pre-grant Pub. No. 2018/0169102, incorporated by reference in its entirety herein. The present disclosure relies on the discovery that administration of an effective amount of trametinib can induce genetic, structural and functional changes associated with neural regeneration and enable survival of differentiated neurons in the ALS animal model. We have further demonstrated that trametinib can enhance motor performance and extend life in the ALS animal model. We have also found that trametinib restores lysosomal activity and induces the fusion of lysosome and autophagosome, thereby protecting axons and dendrites of motor neurons from toxic protein aggregates that cause damage and death in neurons. These results reasonably predict that trametinib will be effective in treating ALS patients.

Since trametinib could induce genetic, structural and functional changes associated with functional recovery of degenerated neurons in general, these data further predict that administration of an effective amount of trametinib could reverse functional defects associated with neurodegenerative diseases and can be used for treatment of ALS as well as other neurodegenerative diseases.

Accordingly, in a first aspect, methods are presented for treating amyotrophic lateral sclerosis (ALS), comprising the step of administering an effective amount of trametinib to a patient diagnosed with ALS.

In some embodiments, trametinib is administered at a daily oral dose effective to induce change in the level of one or more markers in the patient's brain or in a biological sample obtained from the patient of at least 1.3 fold after at least four weeks' administration as compared to prior to administration of trametinib. In some embodiments, the daily oral dose is effective to induce change in the level of the one or more markers in the patient's brain or in a biological sample obtained from the patient of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold.

In some embodiments, trametinib is administered at a daily oral dose effective to decrease the level of one or more markers in the patient's brain or in a biological sample obtained from the patient by at least 20% after the at least four weeks' administration as compared to prior to administration of trametinib. In some embodiments, the daily oral dose is effective to decrease the level of the one or more markers in the patient's brain or in a biological sample obtained from the patient by at least 30%, at least 40%, at least 50%, at least 60%, at least 70%, at least 80%, at least 90%, or at least 99%.

In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. The human homologs include GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcebrosidase, and β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L.

In some embodiments, trametinib is administered at a dose that provides a mean peak trametinib concentration (Cₘₐₓ) of at least 0.25 ng/g in the brain. In some embodiments, trametinib is administered at a dose that provides a mean peak brain trametinib concentration (Cₘₐₓ) is at least 0.5 ng/g in the brain. In some embodiments, trametinib is administered at a dose that provides a mean peak brain trametinib concentration (Cₘₐₓ) of at least 0.75 ng/g in the brain. In some embodiments, trametinib is administered at a dose that provides a mean peak brain trametinib concentration (Cₘₐₓ) of at least 1 ng/g in the brain.

In some embodiments, trametinib is administered at a dose that provides a mean peak brain trametinib concentration (Cₘₐₓ) of at least 1.5 ng/g in the brain. In some embodiments, trametinib is administered at a dose that provides a mean peak brain trametinib concentration (Cₘₐₓ) of at least 2 ng/g in the brain. In some embodiments, trametinib is administered at a dose that provides a mean peak brain trametinib concentration (Cₘₐₓ) of at least 5 ng/g in the brain. In some embodiments, trametinib is administered at a dose that provides a mean peak brain trametinib concentration (Cₘₐₓ) of at least 10 ng/g in the brain. In some embodiments, trametinib is administered at a dose that provides a mean peak brain trametinib concentration (Cₘₐₓ) of at least 15 ng/g in the brain. In some embodiments, trametinib is administered at a dose that provides a mean peak brain trametinib concentration (Cₘₐₓ) of between 0.25 and 20 ng/g in the brain. In some embodiments, trametinib is administered at a dose that provides a mean peak brain trametinib concentration (Cₘₐₓ) of between 0.25 and 5 ng/g in the brain.

In some embodiments, trametinib is administered at an oral dose between 0.5 and 2 mg/day. In some embodiments, trametinib is administered at an oral dose greater than 0.5 and lower than 2 mg/day. In some embodiments, trametinib is administered at an oral dose greater than 0.75 and lower than 2 mg/day. In some embodiments, trametinib is administered at an oral dose greater than 1 and lower than 2 mg/day. In some embodiments, trametinib is administered at an oral dose greater than 0.75 and lower than 1.25 mg/day. In some embodiments, trametinib is administered at an oral dose of 0.5 mg/day. In some embodiments, trametinib is administered at an oral dose of 1 mg/day. In some embodiments, trametinib is administered at an oral dose of 1.5 mg/day. In some embodiments, trametinib is administered at a dose of 2 mg/day.

In some embodiments, trametinib is administered for at least four weeks. In some embodiments, trametinib is administered for at least six weeks. In some embodiments, trametinib is administered for at least seven weeks. In some embodiments, trametinib is administered for at least eight weeks. In some embodiments, trametinib is administered for at least nine weeks. In some embodiments, trametinib is administered for at least three months.

In some embodiments, trametinib is administered in a tablet.

In another aspect, the present disclosure provides a method of treating amyotrophic lateral sclerosis (ALS), comprising the step of administering trametinib to a patient diagnosed with ALS daily, in an amount effective to provide a mean peak trametinib concentration (Cₘₐₓ) of at least 0.25 ng/g in the brain. In some embodiments, the patient does not have BRAF V600E or V600K mutation. In some embodiments, the patient does not have cancer.

In some embodiments, trametinib is administered in an amount effective to induce change in the level of one or more markers in the patient's brain or in a biological sample obtained from the patient of at least 1.3 fold after the at least four weeks compared to before administration of trametinib. In some embodiments, trametinib is administered in an amount effective to induce change in the level of the one or more markers in the patient's brain or in a biological sample obtained from the patient of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold.

In some embodiments, trametinib is administered in an amount effective to decrease the level of one or more markers in the patient's brain or in a biological sample obtained from the patient by at least 20% after the at least four weeks' administration as compared to prior to administration of trametinib. In some embodiments, the amount is effective to decrease the level of the one or more markers in the patient's brain or in a biological sample obtained from the patient by at least 30%, at least 40%, at least 50%, at least 60%, at least 70%, at least 80%, at least 90%, or at least 99%.

In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. The human homologs include GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, DISC1, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRMJ, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcebrosidase, and β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L.

In some embodiments, trametinib is administered at a dose that provides a mean peak trametinib concentration (Cₘₐₓ) of at least 0.25 ng/g in the brain. In some embodiments, trametinib is administered at a dose that provides a mean peak brain trametinib concentration (Cₘₐₓ) is at least 0.5 ng/g in the brain. In some embodiments, the mean peak brain trametinib concentration (Cₘₐₓ) is at least 0.75 ng/g in the brain. In some embodiments, the mean peak brain trametinib concentration (Cₘₐₓ) is at least 1 ng/g in the brain. In some embodiments, the mean peak brain trametinib concentration (Cₘₐₓ) is at least 1.5 ng/g in the brain. In some embodiments, the mean peak brain trametinib concentration (Cₘₐₓ) is at least 2 ng/g in the brain. In some embodiments, the mean peak brain trametinib concentration (Cₘₐₓ) is at least 5 ng/g in the brain. In some embodiments, the mean peak brain trametinib concentration (Cₘₐₓ) is at least 10 ng/g in the brain. In some embodiments, the mean peak brain trametinib concentration (Cₘₐₓ) is at least 15 ng/g in the brain. In some embodiments, the mean peak brain trametinib concentration (Cₘₐₓ) is between 0.25 and 20 ng/g in the brain. In some embodiments, the mean peak brain trametinib concentration (Cₘₐₓ) is between 0.25 and 5 ng/g in the brain.

In some embodiments, trametinib is administered at an oral dose between 0.5 and 2 mg/day. In some embodiments, trametinib is administered at an oral dose greater than 0.5 and lower than 2 mg/day. In some embodiments, trametinib is administered at an oral dose greater than 0.75 and lower than 2 mg/day. In some embodiments, trametinib is administered at an oral dose greater than 1 and lower than 2 mg/day.

In some embodiments, trametinib is administered at an oral dose greater than 0.75 and lower than 1.25 mg/day. In some embodiments, trametinib is administered at an oral dose of 0.5 mg/day. In some embodiments, trametinib is administered at an oral dose of 0.75 mg/day. In some embodiments, trametinib is administered at an oral dose of 1 mg/day. In some embodiments, trametinib is administered at an oral dose of 1.25 mg/day. In some embodiments, trametinib is administered at an oral dose of 1.5 mg/day. In some embodiments, trametinib is administered at an oral dose of 2 mg/day.

In some embodiments, trametinib is administered for at least four weeks. In some embodiments, trametinib is administered for at least six weeks. In some embodiments, trametinib is administered for at least seven weeks. In some embodiments, trametinib is administered for at least eight weeks. In some embodiments, trametinib is administered for at least nine weeks. In some embodiments, trametinib is administered for at least three months.

In some embodiments, the method further comprises the step of testing the level of one or more markers in a sample obtained from the patient. In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. In some embodiments, each of the one or more markers is encoded by a human gene selected from the group consisting of: GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L.

In some embodiments, the sample is obtained after the step of administering trametinib. In some embodiments, the sample is obtained at multiple time points after the step of administering trametinib.

In some embodiments, the method further comprises the step of obtaining the sample. In some embodiments, the method further comprises the step of detecting the level of the one or more markers in a control sample obtained from the patient before the step of administering trametinib. In some embodiments, the method further comprises the step of obtaining the control sample. In some embodiments, the sample is obtained by brain biopsy. In some embodiments, the sample is any biological sample obtained from an individual including body fluids, body tissue, cells, secretions, or other sources. In some embodiments, body fluids or secretions include blood, urine, saliva, stool, pleural fluid, lymphatic fluid, sputum, ascites, prostatic fluid, cerebrospinal fluid (CSF), or any other bodily secretion or derivative thereof. In some embodiments, blood is selected from whole blood, plasma, serum, peripheral blood mononuclear cells (PBMC), or any components of blood.

In some embodiments, the method further comprises the step of determining therapeutic efficiency of trametinib administered to the patient based on the level of the one or more markers. In some embodiments, the method further comprises the step of determining duration or dose for subsequent administration of trametinib. In some embodiments, the method further comprises the step of discontinuing administration of trametinib based on determination of the therapeutic efficacy. In some embodiments, the method further comprises the step of continuing administration of trametinib based on determination of the therapeutic efficacy. In some embodiments, the method further comprises the step of adjusting administration of trametinib based on determination of the therapeutic efficacy.

In some embodiments, the method further comprises: (a) detecting the level of the one or more markers in a biological sample obtained from the patient following administration of trametinib; and (b) comparing the level detected in (a) with the level of the one or more markers in a biological sample obtained from the patient prior to administration of trametinib; or (c) comparing the level detected in (a) with the level of the one or more markers in a biological sample obtained from the healthy subjects who are free of the disease(s) of interest.

In yet another aspect, the present disclosure provides a method of enhancing lysosomal activity in a target tissue, comprising the step of administering trametinib to a subject, wherein the subject was diagnosed with a disorder associated with lysosomal dysfunction or autophagic flux. In some embodiments, the disorder is selected from the group consisting of: lysosome storage disease, Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, neuronal intranuclear hyaline inclusion disease, multiple sclerosis, glaucoma and age-related macular degeneration.

In some embodiments, the lysosome storage disorder is selected from the group consisting of: alpha-mannosidosis, aspartylglucosaminuria, juvenile Neuronal Ceroid Lipofuscinosis (JNCL, juvenile Batten or CLN3 Disease), cystinosis, Fabry Disease, Gaucher Disease Types I, II, and III, Glycogen Storage Disease II (Pompe Disease), GM2-Gangliosidosis Type I (Tay Sachs Disease), GM2-Gangliosidosis Type II (Sandhoff Disease), Metachromatic Leukodystrophy, Mucolipidosis Types I, II/III and IV, Mucopolysaccharide Storage Diseases (Hurler Disease and variants, Hunter, Sanfilippo Types A,B,C,D, Morquio Types A and B, Maroteaux-Lamy and Sly diseases), Niemann-Pick Disease Types A/B, C1 and C2, Schindler Disease Types I and II.

In some embodiments, trametinib is administered for at least four weeks. In some embodiments, trametinib is administered for at least five weeks. In some embodiments, trametinib is administered for at least six weeks. In some embodiments, trametinib is administered for at least seven weeks. In some embodiments, trametinib is administered for at least eight weeks. In some embodiments, trametinib is administered for at least nine weeks. In some embodiments, trametinib is administered for at least three months.

In some embodiments, trametinib is administered at a daily oral dose effective to induce change in the level of one or more markers in the patient's target tissue or in a biological sample obtained from the patient of at least 1.3 fold after the at least four weeks' administration compared to prior to administration of trametinib. In some embodiments, the daily oral dose is effective to induce change in the level of the one or more markers in the patient's target tissue or in a biological sample obtained from the patient of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold.

In some embodiments, trametinib is administered at a daily oral dose effective to decrease the level of one or more markers in the patient's brain or in a biological sample obtained from the patient by at least 20% after the at least four weeks' administration as compared to prior to administration of trametinib. In some embodiments, the daily oral dose is effective to decrease the level of the one or more markers in the patient's brain or in a biological sample obtained from the patient by at least 30%, at least 40%, at least 50%, at least 60%, at least 70%, at least 80%, at least 90%, or at least 99%.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L.

In some embodiments, trametinib administration provides a mean peak trametinib concentration (Cₘₐₓ) of at least 0.25 ng/g in the target tissue. In some embodiments, the mean peak trametinib concentration (Cₘₐₓ) is at least 0.5 ng/g in the target tissue. In some embodiments, the mean peak trametinib concentration (Cₘₐₓ) is at least 0.75 ng/g in the target tissue. In some embodiments, the mean peak trametinib concentration (Cₘₐₓ) is at least 1 ng/g in the target tissue. In some embodiments, the mean peak trametinib concentration (Cₘₐₓ) is at least 1.5 ng/g in the target tissue. In some embodiments, the mean peak trametinib concentration (Cₘₐₓ) is at least 2 ng/g in the target tissue. In some embodiments, the mean peak trametinib concentration (Cₘₐₓ) is at least 5 ng/g in the target tissue. In some embodiments, the mean peak trametinib concentration (Cₘₐₓ) is at least 10 ng/g in the target tissue. In some embodiments, the mean peak trametinib concentration (Cₘₐₓ) is at least 15 ng/g in the target tissue. In some embodiments, the mean peak trametinib concentration (Cₘₐₓ) is between 0.25 and 20 ng/g in the target tissue. In some embodiments, the mean peak trametinib concentration (Cₘₐₓ) is between 0.25 and 5 ng/g in the target tissue.

In some embodiments, the target tissue is brain. In some embodiments, the target tissue is spinal cord.

In some embodiments, trametinib is administered at an oral dose between 0.5 and 2 mg/day. In some embodiments, trametinib is administered at an oral dose greater than 0.5 and lower than 2 mg/day. In some embodiments, trametinib is administered at an oral dose greater than 0.75 and lower than 2 mg/day. In some embodiments, trametinib is administered at an oral dose greater than 1 and lower than 2 mg/day. In some embodiments, trametinib is administered at an oral dose greater than 0.75 and lower than 1.25 mg/day. In some embodiments, trametinib is administered at an oral dose of 0.5 mg/day. In some embodiments, trametinib is administered at an oral dose of 1 mg/day. In some embodiments, trametinib is administered at an oral dose of 1.5 mg/day. In some embodiments, trametinib is administered at a dose of 2 mg/day.

### 4. BRIEF DESCRIPTION OF THE DRAWINGS

FIG. 1 shows brain (top) and plasma (bottom) concentration-time profiles of trametinib after single oral administration of trametinib in mice.

FIG. 2 provides a representative image of western blot analysis of pERKs and ERKs in mice whole brain lysates. ERKs were included as a loading control.

FIGS. 3A and 3B show up-regulated genes (FIG. 3A) and down-regulated genes (FIG. 3B), that are relevant to various biological processes in the brain of normal mice administered with trametinib, with each panel showing data from a different timepoint. The biological processes are defined by the ontology of enriched genes.

FIGS. 4A and 4B illustrate genes involved in synaptic activity, neurogenesis, lysosomal activity and autophagosome activity showing significant mRNA expression level changes by administration of trametinib compared to the vehicle treated group. The values in FIG. 4B represent fold change (FC) in the mRNA expression levels of the trametinib treated group compared to those of the vehicle treated group.

FIG. 5 shows changes in grip strength by days of administration of vehicle or trametinib at 0.1 or 0.2 mg/kg (“mpk”) in the ALS model mice or in wild type mice.

FIG. 6 shows changes in grip strength at day 10 of administration of vehicle or trametinib (0.1 or 0.2 mpk) in the ALS model mice or in wild type mice.

FIG. 7 is a histogram showing changes in motor performance scores by days of administration of vehicle or trametinib (0.1 or 0.2 mpk) in the ALS model mice or in wild type mice. # denotes that both the 0.1 and 0.2 mg/kg/day groups showed significant differences (p<0.05) compared to the vehicle-treated group; * denotes that the 0.2 mg/kg/day group showed a significant difference (p<0.05) compared to the vehicle-treated group.

FIG. 8 shows changes in motor performance scores at day 11 of administration of vehicle or trametinib (0.1 or 0.2 mpk) in the ALS model mice or in wild type mice.

FIG. 9 shows changes in numbers of surviving mice by days of administration of vehicle or trametinib (0.1 or 0.2 mpk) in the ALS model mice or in wild type mice.

FIG. 10 provides images of Nissl staining of the spinal cord tissues of wild type and vehicle-treated and trametinib-treated ALS model mice.

FIG. 11 provides immunofluorescence staining (staining for SMI-32 and NeuN) images of motor neurons in wild type mice, and vehicle or trametinib-treated ALS model mice.

FIG. 12 provides immunofluorescence staining (staining for Longitudinal Tau) images of axonal tracts in wild type mice, and vehicle or trametinib-treated ALS model mice.

FIG. 13A provides immunofluorescence staining (staining for Tuj 1) images of axons in wild type mice, and vehicle or trametinib-treated ALS model mice. FIG. 13B is a bar graph of the axonal length in each group.

FIG. 14A provides immunofluorescence staining (staining for MAP2) images of dendrites in wild type mice, and vehicle or trametinib-treated ALS model mice. FIG. 14B is a bar graph of the dendritic length in each group.

FIG. 15A provides immunofluorescence staining images of lysosomes and autophagosomes in wild type mice, and vehicle or trametinib-treated ALS model mice. Images from immunostaining against LC3 (in the middle) and LAMP1 (on the bottom) are provided, and their merged images are provided in the top. FIG. 15B is a bar graph showing quantification of LC3 and LAMP1 co-localization in each group.

The figures depict various embodiments of the present invention for purposes of illustration only. One skilled in the art will readily recognize from the following discussion that alternative embodiments of the structures and methods illustrated herein may be employed without departing from the principles of the invention described herein.

### 5. DETAILED DESCRIPTION

### 5.1. Definitions

Unless defined otherwise, all technical and scientific terms used herein have the meaning commonly understood by a person skilled in the art to which this invention belongs. As used herein, the following terms have the meanings ascribed to them below.

The term “MEK 1/2 inhibitor” as used herein refers to a compound that inhibits the function of both MEK 1 and MEK 2.

An exemplary MEK 1/2 inhibitor is trametinib (GSK-1120212, GSK1120212, JTP74057, or JTP-74057). The chemical name for trametinib is acetamide, N-[3-[3-cyclopropyl-5-[(2-fluoro-4-iodophenyl)amino]-3,4,6,7-tetrahydro-6,8-dimethyl-2,4,7-trioxopyrido[4,3-d]pyrimidin-1(2H)-yl]phenyl]. It has a molecular formula C₂₆H₂₃FIN₅O₄ with a molecular mass of 615.39. Trametinib has the chemical structure of Formula 1.

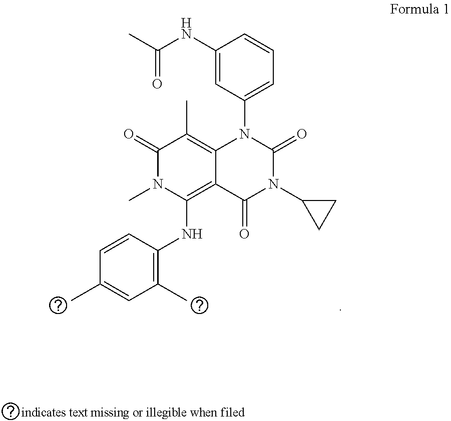 [SMILES: CC(=O)Nc1cccc(-n2c(=O)n(C3CC3)c(=O)c3c(Nc4ccc(C)cc4C)n(C)c(=O)c(C)c32)c1]

In the commercially available product, MEKINIST®, trametinib is in the form a dimethyl sulfoxide solvate. In the methods described herein, trametinib can be used in the form of a free base or a pharmaceutically acceptable salt or solvate, including the dimethyl sulfoxide solvate. Examples of possible solvates are hydrates, dimethyl sulfoxide, acetic acid, ethanol, nitromethane, chlorobenzene, 1-pentanol, isopropyl alcohol, ethylene glycol, 3-methyl-1-butanol, etc.

The term “therapeutically effective dose” or “effective amount” as used herein refers to a dose or amount that produces the desired effect for which it is administered. In the context of the present methods, a therapeutically effective amount is an amount effective to treat a symptom or improve a disease state of a subject with a neurodegenerative disease, in particular, ALS. The term “sufficient amount” as used herein refers to an amount sufficient to produce a desired effect.

### 5.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 5.3. Methods of Treating a Neurodegenerative Disease

In a first aspect, methods are presented for treating a patient with a neurodegenerative disease, e.g., ALS patients. The method comprises administering an effective amount of trametinib.

In some embodiments, trametinib is administered daily for at least four weeks. In some embodiments, trametinib is administered to provide a mean peak trametinib concentration (Cₘₐₓ) of at least 0.25 ng/g in the brain. Various delivery methods can be used to administer trametinib in the methods described herein. In currently preferred embodiments, trametinib is delivered by oral administration.

### 5.3.1. Subject for Treatment with Trametinib

5.3.1.1. Patients with ALS

In various embodiments, the patient has sporadic ALS. In various embodiments, the patient has familial ALS.

In some embodiments, patients with early-onset ALS are treated. In some embodiments, patients with late-onset ALS are treated.

In some embodiments, patients with respiratory-onset ALS are treated. In some embodiments, patients with spinal-onset or bulbar-onset ALS are treated.

In some embodiments, ALS patients with a specific mutation in a gene involved in protein degradation, the cytoskeleton, or RNA processing are treated. In particular embodiments, the method further comprises determining, prior to treatment, whether the ALS patient has the specific mutation.

In certain of these embodiments, ALS patients with a specific mutation in FUS or SOD1 are treated. In some embodiments, ALS patients with a specific mutation in ALS2, SETX, SPG11, FUS, or SIGMAR1 are treated. In some embodiments, ALS patients with a specific mutation in VCP, OPTN, TBK1, or SQSTM1 are treated. In some embodiments, ALS patients with a specific mutation in DCTN1, PFN1, or TUBA4A are treated. In some embodiments, ALS patients with a specific mutation in TARDBP, the gene that codes for TDP-43, are treated. In some embodiments, ALS patients with a specific mutation in TARDBP or FUS are treated. In some embodiments, ALS patients with a specific mutation in ANG, SETX, or MATR3 are treated. In some embodiments, ALS patients with a specific mutation in C9orf72 are treated. In some embodiments, ALS patients with a specific mutation in excitatory amino acid transporter 2 (EAAT2) are treated.

In some embodiments, patients with primary lateral sclerosis (PLS) are treated. In some embodiments, patients with progressive muscular atrophy (PMA) are treated. In some embodiments, patients with a regional variant of ALS are treated. In certain embodiments, the patients have flail arm syndrome (brachial amyotrophic diplegia), flail leg syndrome (leg amyotrophic diplegia), or isolated bulbar ALS.

In some embodiments, patients are treated before having any visible symptoms of ALS. In some embodiments, patients with initial symptoms are treated. In some embodiments, ALS patients with progression of the disease are treated. In some embodiments, ALS patients at late stage of the disease are treated.

5.3.1.2. Patients with a Neurodegenerative Disease

In the Examples below, we demonstrate that trametinib has multifaceted therapeutic actions that promote the functional recovery of degenerated cerebral neurons. Accordingly, the method described herein can be used for treatment of neurodegenerative diseases characterized by cortical degeneration.

Neurodegenerative diseases that can be treated with the methods provided herein include, but are not limited to, dementia, vascular dementia, senile dementia, frontotemporal dementia (FTD), Lewy body dementia (LBD), Parkinson's disease (PD), multiple system atrophy (MSA), corticobasal degeneration (CBD), progressive supranuclear palsy (PSP), Huntington's disease (HD), amyotrophic lateral sclerosis (ALS, Lou-Gehrig's disease), primary lateral sclerosis (PLS), progressive bulbar palsy (PBP), progressive muscular atrophy (PMA), pseudobulbar palsy, hereditary spastic paraplegia (HSP), cerebellar ataxia, Creutzfeldt-Jakob disease (CJD), multiple sclerosis (MS), Guillain-Barré syndrome (GBS), and mild cognitive impairment (MCI).

In some embodiments, the patient has one or more symptoms, such as memory loss, language problems, unpredictable behavior, and personality and behavioral changes. In some embodiments, the patient does not have any behavioral symptom. In some embodiments, the patient has changes in one or more biomarkers associated with a neurodegenerative disease.

In some embodiments, the patient has mild cognitive impairment (MCI). In some embodiments, the patient has memory complaints and memory difficulties. In some embodiments, the patient has abnormal memory function documented by scoring below the education adjusted cutoff on the Logical Memory II subscale (Delayed Paragraph Recall) from the Wechsler Memory Scale—Revised (the maximum score is 25): a) less than or equal to 8 for 16 or more years of education, b) less than or equal to 4 for 8-15 years of education, c) less than or equal to 2 for 0-7 years of education. In some embodiments, the patient has Mini-Mental Exam score between 24 and 30 (inclusive). In some embodiments, the patient's Clinical Dementia Rating is 0.5 and Memory Box score is at least 0.5. In some embodiments, the patient has general cognition and functional performance sufficiently preserved such that a diagnosis of AD cannot be made.

In some embodiments, the neurodegenerative disease involves abnormal activation of MAPK. In some embodiments, the neurodegenerative disease involves abnormal endosomal-lysosomal function. In some embodiments, the neurodegenerative disease is Niemann-Pick Type C (NPC).

In preferred embodiments, the patient does not have BRAF V600E or V600K mutation and the patient does not have cancer.

5.3.1.3. Patients with a Disorder Associated with Lysosomal Dysfunction or Autophagic Flux

In the Examples below, we also show that trametinib facilitates lysosomal activity; accordingly, trametinib can be used in the treatment of diseases characterized by lysosomal dysfunction or autophagic flux dysfunction. Therefore, trametinib can be used in the treatment of diseases characterized by lysosomal dysfunction or autophagic flux dysfunction. Such diseases include, but are not limited to, lysosome storage disease, amyotrophic lateral sclerosis (ALS), Alzheimer's disease, Parkinson's disease, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, neuronal intranuclear hyaline inclusion disease, multiple sclerosis, glaucoma and age-related macular degeneration. Lysosomal storage disease includes, but not limited to, alpha-mannosidosis, aspartylglucosaminuria, juvenile Neuronal Ceroid Lipofuscinosis (JNCL, juvenile Batten or CLN3 Disease), cystinosis, Fabry Disease, Gaucher Disease Types I, II, and III, Glycogen Storage Disease II (Pompe Disease), GM2-Gangliosidosis Type I (Tay Sachs Disease), GM2-Gangliosidosis Type II (Sandhoff Disease), Metachromatic Leukodystrophy, Mucolipidosis Types I, II/III and IV, Mucopolysaccharide Storage Diseases (Hurler Disease and variants, Hunter, Sanfilippo Types A,B,C,D, Morquio Types A and B, Maroteaux-Lamy and Sly diseases), Niemann-Pick Disease Types A/B, C1 and C2, Schindler Disease Types I and II.

### 5.3.2. Administration of Trametinib

5.3.2.1. Dose

The selected patient is administered a therapeutically effective amount of trametinib daily. In the methods described herein, the therapeutically effective dose is a dose effective to treat a neurodegenerative disease (e.g., ALS) in the subject. In a particular embodiment, the therapeutically effective dose is a dose effective to treat ALS in the subject.

In some embodiments, the therapeutically effective dose is the dose sufficient to induce neural differentiation. In some embodiments, the therapeutically effective dose is the dose sufficient to induce neural regeneration. In some embodiments, the therapeutically effective dose is the dose sufficient to induce lysosomal activity. In some embodiments, the therapeutically effective dose is the dose sufficient to enhance autophagosome-lysosome fusion in the subject. In some embodiments, the therapeutically effective dose is the dose sufficient to protect newly formed axons in the nervous system.

In some embodiments, the therapeutically effective dose is the dose sufficient to induce expression of genes involved in synaptic formation in the brain. In some embodiments, the therapeutically effective dose is the dose sufficient to induce expression of genes involved in neuroblast proliferation in the brain. In some embodiments, the therapeutically effective dose is the dose sufficient to induce expression of genes involved in axon growth in the brain. In some embodiments, the therapeutically effective dose is the dose sufficient to induce expression of genes involved in enhancing lysosomal activity. In some embodiments, the therapeutically effective dose is the dose sufficient to induce expression of genes involved in immune response in the brain. In some embodiments, the therapeutically effective dose is the dose sufficient to induce expression of genes involved in lysosomal activity. In some embodiments, the therapeutically effective dose is the dose sufficient to induce expression of genes involved in synaptic formation, neuroblast proliferation, axon growth, and lysosomal activity in the brain.

In some embodiments, trametinib is administered in a dose sufficient to induce change in the level of one or more markers. In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. In some embodiments, each of the one or more markers is encoded by a human gene selected from the group consisting of: GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L. The proteins can be used as a marker protein for measuring effects of trametinib.

In some embodiments, trametinib is administered at a dose effective to induce change in the level of one or more markers in the patient's brain or in a biological sample obtained from the patient of at least 1.3 fold, at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold after at least four weeks' administration as compared to prior to administration of trametinib. In some embodiments, trametinib is administered at a dose effective to induce change in the level of one or more markers in the patient's brain or in a biological sample obtained from the patient of at least 1.3 fold, at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold after four weeks', eight weeks', ten weeks' twelve weeks', or twenty-four weeks' administration as compared to prior to administration of trametinib.

In some embodiments, trametinib is administered at a dose effective to decrease the level of one or more markers in the patient's brain or in a biological sample obtained from the patient by at least 20%, at least 30%, at least 40%, at least 50%, at least 60%, at least 70%, at least 80%, at least 90%, or at least 99% after at least four weeks' administration as compared to prior to administration of trametinib. In some embodiments, trametinib is administered at a dose effective to decrease the level of one or more markers in the patient's brain or in a biological sample obtained from the patient by at least 20%, at least 30%, at least 40%, at least 50%, at least 60%, at least 70%, at least 80%, at least 90%, or at least 99% after four weeks′, eight weeks′, ten weeks′, twelve weeks′, or twenty-four weeks' administration as compared to prior to administration of trametinib.

In some embodiments, trametinib is administered at a dose that provides a mean peak trametinib concentration (Cₘₐₓ) of at least 0.25 ng/g in the brain. In some embodiments, trametinib is administered at a dose that provides a mean peak trametinib concentration (Cₘₐₓ) of at least 0.5, 0.75, 1, 1.25, 1.50, 1.75, or 2 ng/g in the brain. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 ng/g in the brain. In some embodiments, trametinib is administered at a dose that provides a mean peak trametinib concentration (Cₘₐₓ) of about 4, 5, 6, 7, 8, 9, 10, 11, 12, 13, 14, 15, 16, 17, 18, 19 or 20 ng/g in the brain. In some embodiments, trametinib is administered at a dose that provides a mean peak trametinib concentration (Cₘₐₓ) of between 0.25 and 20, between 0.25 and 10, between 0.25 and 5, between 0.5 and 5, between 2.5 and 10, between 1 and 5 ng/g in the brain.

In some embodiments, trametinib is administered at a dose that provides a mean peak trametinib concentration (Cₘₐₓ) of at least 0.25 ng/ml in CSF. In some embodiments, trametinib is administered at a dose that provides a mean peak trametinib concentration (Cₘₐₓ) of at least 0.5, 0.75, 1, 1.25, 1.50, 1.75, or 2 ng/ml in CSF. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 ng/ml in CSF. In some embodiments, trametinib is administered at a dose that provides a mean peak trametinib concentration (Cₘₐₓ) of about 4, 5, 6, 7, 8, 9, 10, 11, 12, 13, 14, 15, 16, 17, 18, 19 or 20 ng/ml in CSF. In some embodiments, trametinib is administered at a dose that provides a mean peak trametinib concentration (Cₘₐₓ) of between 0.25 and 20, between 0.25 and 10, between 0.25 and 5, between 0.5 and 5, between 2.5 and 10 ng/ml, between 1 and 5 ng/ml in CSF.

In some embodiments, trametinib is administered at a dose that provides a mean peak trametinib concentration (Cₘₐₓ) of no more than 4.4 ng/g in the brain. In some embodiments, trametinib is administered at a dose that provides a mean peak trametinib concentration (Cₘₐₓ) of no more than 2 ng/g in the brain. In some embodiments, trametinib is administered at a dose that provides a mean peak trametinib concentration (Cₘₐₓ) of no more than 1.8 ng/g, no more than 1.6 ng/g, no more than 1.4 ng/g, no more than 1.2 ng/g, no more than 1 ng/g, no more than 0.8 ng/g, no more than 0.6 ng/g, or no more than 0.4 ng/g in the brain.

In some embodiments, trametinib is administered at a dose that provides a mean peak trametinib concentration (Cₘₐₓ) of no more than 4.4 ng/ml in CSF. In some embodiments, trametinib is administered at a dose that provides a mean peak trametinib concentration (Cₘₐₓ) of no more than 2 ng/ml in CSF. In some embodiments, trametinib is administered at a dose that provides a mean peak trametinib concentration (Cₘₐₓ) of no more than 1.8 ng/ml, no more than 1.6 ng/ml, no more than 1.4 ng/ml, no more than 1.2 ng/ml, no more than 1 ng/g, no more than 0.8 ng/ml, no more than 0.6 ng/ml, or no more than 0.4 ng/ml in CSF.

In some embodiments, trametinib is administered at a dose that provides a mean peak trametinib concentration (Cₘₐₓ) of no more than 22.2 ng/ml in the plasma. In some embodiments, trametinib is administered at a dose that provides a mean peak trametinib concentration (Cₘₐₓ) of no more than 22 ng/ml in the plasma. In some embodiments, trametinib is administered at a dose that provides a mean peak trametinib concentration (Cₘₐₓ) of no more than 20 ng/ml, no more than 18 ng/ml, no more than 16 ng/ml, no more than 14 ng/ml, no more than 12 ng/ml, no more than 10 ng/ml, no more than 8 ng/ml, or no more than 6 ng/ml in the plasma.

In some embodiments, trametinib is administered at a dose that provides an area under the concentration curve (AUC) of trametinib in the brain of at least 25, 30, 35, 40, 45, 50, 55, 60, 65, 70, 75, 80, 85, 90, or 100 ng·h/g. In some embodiments, trametinib is administered at a dose that provides an area under the brain concentration curve of trametinib of about 25, 30, 35, 40, 45, 50, 55, 60, 65, 70, 75, 80, 85, 90, 95, 100, 110, 120, 130, 140, 150, 160, 170, 180, 190, 200, 250, or 300 ng·h/g. In some embodiments, trametinib is administered at a dose that provides an area under the brain concentration curve of trametinib of about 20 to about 700 ng·h/g, about 20 to about 600 ng·h/g, about 30 to about 500 ng·h/g, about 50 to about 400 ng·h/g, about 50 to about 300 ng·h/g, about 50 to about 200 ng·h/g, about 50 to about 100 ng·h/g, about 60 to 300 ng·h/g.

In some embodiments, trametinib is administered at a dose that provides an area under the concentration curve (AUC) of trametinib in CSF of at least 25, 30, 35, 40, 45, 50, 55, 60, 65, 70, 75, 80, 85, 90, or 100 ng·h/ml. In some embodiments, trametinib is administered at a dose that provides an area under the CSF concentration curve of trametinib of about 25, 30, 35, 40, 45, 50, 55, 60, 65, 70, 75, 80, 85, 90, 95, 100, 110, 120, 130, 140, 150, 160, 170, 180, 190, 200, 250, or 300 ng·h/ml. In some embodiments, trametinib is administered at a dose that provides an area under the CSF concentration curve of trametinib of about 20 to about 700 ng·h/ml, about 20 to about 600 ng·h/ml, about 30 to about 500 ng·h/ml, about 50 to about 400 ng·h/ml, about 50 to about 300 ng·h/ml, about 50 to about 200 ng·h/ml, about 50 to about 100 ng·h/ml, about 60 to 300 ng·h/ml. In some embodiments, trametinib is administered at a dose that provides an area under the CSF concentration curve of trametinib of about 30 to about 200 ng·h/ml.

In some embodiments, trametinib is administered at a dose that provides a mean peak trametinib concentration (Cₘₐₓ) of at least 0.25 ng/ml in the plasma. In some embodiments, trametinib is administered at a dose that provides a mean peak trametinib concentration (Cₘₐₓ) of at least 0.5, 0.75, 1, 1.25, 1.50, 1.75, 2, 2.25, 2.50, 2.75, 3, 3.25, 3.50, 3.75, 4, 4.25, 4.50, 4.75, or 5 ng/ml in the plasma. In some embodiments, trametinib is administered at a dose that provides a mean peak trametinib concentration (Cₘₐₓ) of at least 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6.0 ng/ml in the plasma. In some embodiments, trametinib is administered at a dose that provides a mean peak trametinib concentration (Cₘₐₓ) of about 4, 5, 6, 7, 8, 9, 10, 11, 12, 13, 14, 15, 16, 17, 18, 19, 20, 21, 22, 23, 24, 25, 26, 27, 28, 29, 30, or 40 ng/ml in the plasma. In some embodiments, trametinib is administered at a dose that provides a mean peak trametinib concentration (Cₘₐₓ) of between 1 and 400, between 2 and 400, between 1 and 200, between 1 and 150, between 1 and 100, between 2 and 100, between 3 and 100, between 4 and 100, between 5 and 100, between 10 and 100, between 15 and 100, between 15 and 90, between 20 and 80, between 2.5 and 50, between 2.5 and 25, between 2.5 and 10 ng/ml, between 3 and 50 ng/ml in the plasma.

In some embodiments, trametinib is administered at a dose that provides an area under the plasma concentration curve of trametinib of at least 25, 30, 35, 40, 45, 50, 55, 60, 65, 70, 75, 80, 85, 90, 95, 100, 110, 120, 130, 140, 150, 160, 170, 180, 190 or 200 ng·h/mL. In some embodiments, trametinib is administered at a dose that provides an area under the plasma concentration curve of trametinib of about 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 400, or 500 ng·h/mL. In some embodiments, trametinib is administered at a dose that provides an area under the plasma concentration curve of trametinib of about 20 to about 700 ng·h/mL, about 20 to about 600 ng·h/mL, about 30 to about 500 ng·h/mL, about 50 to about 400 ng·h/mL, about 50 to about 300 ng·h/mL, about 50 to about 200 ng·h/mL, about 50 to about 100 ng·h/mL, about 100 to about 500 ng·h/mL.

In some embodiments, trametinib is administered at a dose between 0.5 and 2 mg/day. In some embodiments, trametinib is administered at a dose between 0.75 and 2 mg/day. In some embodiments, trametinib is administered at a dose between 1 and 2 mg/day. In some embodiments, trametinib is administered at a dose between 0.75 and 1.25 mg/day. In some embodiments, trametinib is administered at a dose between 0.5 and 1 mg/day. In some embodiments, trametinib is administered at a dose of 0.5 mg/day. In some embodiments, trametinib is administered at a dose of 1 mg/day. In some embodiments, trametinib is administered at a dose of 1.5 mg/day. In some embodiments, trametinib is administered at a dose of 2 mg/day.

In some embodiments, trametinib is administered at a dose greater than 0.5 mg/day and lower than 2 mg/day. In some embodiments, trametinib is administered at a dose greater than 0.75 mg/day and lower than 2 mg/day. In some embodiments, trametinib is administered at a dose greater than 1 mg/day and lower than 2 mg/day. In some embodiments, trametinib is administered at a dose greater than 0.75 mg/day and lower than 1.25 mg/day. In some embodiments, trametinib is administered at a dose greater than 0.5 and lower than 1 mg/day.

In a preferred embodiment, each dose is a daily dose delivered as a single oral uptake. In some embodiments, each dose is divided into several oral uptakes. In some embodiments, each dose is divided into equal uptake doses. In some embodiments, each dose is divided into unequal uptake doses. In preferred embodiments, each dose is administered at regular intervals.

5.3.2.2. Duration

In some embodiments, trametinib is administered for a period sufficient to induce neural differentiation. In some embodiments, trametinib is administered for a period sufficient to induce neural regeneration. In some embodiments, trametinib is administered for a period sufficient to induce lysosomal activity. In some embodiments, trametinib is administered for a period sufficient to induce axonogenesis.

In certain embodiments, trametinib is administered for at least four weeks, for at least five weeks, for at least six weeks, for at least seven weeks, for at least eight weeks, for at least nine weeks, or for at least ten weeks. In certain embodiments, trametinib is administered for at least one month, for at least two months, for at least three months, or for at four months. In some embodiments, trametinib is administered for about six weeks, seven weeks, eight weeks, nine weeks, ten weeks or more. In some embodiments, trametinib is administered for about one month, two months, three months, four months, five months, six months, or more.

In some embodiments, trametinib is administered for five to six weeks, six to seven weeks, seven to eight weeks, eight to nine weeks, nine to ten weeks, ten to eleven weeks, eleven to twelve weeks, or twelve to thirteen weeks. In some embodiments, trametinib is administered for one to two months, two to three months, three to four months, four to five months, five to six months, six to seven months, seven to eight months, eight to nine months, nine to ten months, ten to eleven months, or eleven to twelve months.

In preferred embodiments, trametinib is administered for a period sufficient to induce neural differentiation in the subject. In some embodiments, trametinib is administered for a period sufficient to enhance lysosomal activity in the subject. In some embodiments, trametinib is administered for a period sufficient to enhance autophagosome-lysosome fusion in the subject. In some embodiments, trametinib is administered for a period sufficient to protect newly formed axons in the nervous system.

In some embodiments, trametinib is administered for a period sufficient to induce expression of genes involved in synaptic formation in the brain. In some embodiments, trametinib is administered for a period sufficient to induce expression of genes involved in neuroblast proliferation in the brain. In some embodiments, trametinib is administered for a period sufficient to induce expression of genes involved in axon growth in the brain. In some embodiments, trametinib is administered for a period sufficient to induce expression of genes involved in immune response in the brain. In some embodiments, trametinib is administered for a period sufficient to induce expression of genes involved in lysosomal activity. In some embodiments, trametinib is administered for a period sufficient to induce expression of genes involved in synaptic formation, neuroblast proliferation, axon growth, and lysosomal activity in the brain.

In some embodiments, trametinib is administered until change in the level of one or more markers is detected. In some embodiments, each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. In some embodiments, each of the one or more markers is encoded by a human gene selected from the group consisting of: GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. In some embodiments, the protein related to lysosomal activity is glycohydrolase or protease. In some embodiments, the glycohydrolase is selected from the group consisting of: β-hexosaminidase, β-galactosidase, β-galactosylcerebrosidase, β-glucuronidase. In some embodiments, the protease is cathepsin. In some embodiments, the cathepsin is selected from the group consisting of: Cathepsin S, Cathepsin D, Cathepsin B, Cathepsin K, and Cathepsin L. The proteins can be used as a marker protein for measuring effects of trametinib.

In some embodiments, trametinib is administered until the level of one or more markers reaches more than at least 1.3×, 1.5×, 2×, 3×, 4×, 5×, 6×, 7×, 8×, 9×, 10×20×, 30×, 40×, 50×, 100×, 200×, or 1000× of the levels measured prior to or without administration of trametinib. In some embodiments, trametinib is administered until the level of one or more markers reaches less than at most 0.8×, 0.7×, 0.6×, 0.5×, 0.4×, 0.3×, 0.2×, 0.1×, 0.05×, or 0.01× of the levels measured prior to or without administration of trametinib. In some embodiments, trametinib is administered until the level of one or more markers in patient's brain or in a biological sample obtained from the patient decrease by at least 20%, at least 30%, at least 40%, at least 50%, at least 60%, at least 70%, at least 80%, at least 90%, or at least 99% compared to the levels measured prior to or without administration of trametinib.

In some embodiments, trametinib is administered until the level of one or more markers reaches more than at least 1.3×, 1.5×, 2×, 3×, 4×, 5×, 6×, 7×, 8×, 9×, 10×, 20×, 30×, 40×, 50×, 100×, 200×, or 1000× of a fixed or predetermined level. In some embodiments, trametinib is administered until the level of one or more markers reaches less than at most 0.8×, 0.7×, 0.6×, 0.5×, 0.4×, 0.3×, 0.2×, 0.1×, 0.05×, or 0.01× of a fixed or predetermined level. In some embodiments, trametinib is administered until the level of one or more markers in patient's brain or in a biological sample obtained from the patient decrease by at least 20%, at least 30%, at least 40%, at least 50%, at least 60%, at least 70%, at least 80%, at least 90%, or at least 99% compared to a fixed or predetermined level.

In some embodiments, trametinib is administered until a desired therapeutic outcome is detected. In some embodiments, the desired therapeutic outcome is change in behavioral or physiological symptom of the patient. In some embodiments, trametinib is administered until unacceptable toxicity occurs.

### 5.4. Detection of Markers

In another aspect, a method of testing the therapeutic outcome of a drug (e.g., MEK 1/2 inhibitor such as trametinib) in a neurodegenerative subject is provided. The method involves the step of measuring the level of one or more markers in a sample obtained from the subject.

In some embodiments, the method of treating a neurodegenerative disease provided herein further comprises the step of testing expression of one or more markers in a sample obtained from the subject. Expression of one or more markers can be tested using a method known in the art by measuring proteins or by measuring mRNAs, using methods such as western blotting, ELISA, RT-PCR, qPCR, immunoelectrophoresis, protein immunoprecipitation, and protein immunostaining. Various methods of measuring amounts of mRNA or proteins can be adopted for the method.

In some embodiments, the method of treating a neurodegenerative disease provided herein further comprises the step of measuring the level of one or more marker proteins in a sample obtained from the subject. Level of one or more marker proteins can be measured using various protein assays known in the art. For example, the sample may be contacted with an antibody specific for said marker under conditions sufficient for an antibody-marker complex to form, and then detecting said complex. The presence of the protein marker may be detected in a number of ways, such as western blotting, ELISA, immunoelectrophoresis, protein immunoprecipitation, protein immunostaining, 2-dimensional SDS-PAGE, fluorescence activated cell sorting (FACS), and flow cytometry.

The level of one or more markers can be measured at multiple time points, and the amounts measured at different time points can be compared. Changes in the level of one or more markers over time can be used to determine therapeutic effects of an MEK 1/2 inhibitor such as trametinib in the patient.

In some embodiments, each of the one or more markers is encoded by a human homolog of the mouse gene selected from the group consisting of Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. In some embodiments, each of the one or more markers is encoded by a human gene selected from the group consisting of: GABRB1, GABRR2, GLRA3, NR3C2, CDKL5, GRIN2A, GRIN2B, PLCXD3, CHRM2, CHRNA3, CHRNA7, CHRNB2, NEFL, PLD1, ADRA1A, CHRNB3, SLC6A3, SLC18A2, CDH1, NEUROD1, NKX6-1, CXCL6, REST, SYT2, IRX3, MDM4, SOX14, GRIP1, PAX2, BMP5, CPNE1, NUMB, ATP8A2, TRIM67, OTP, IL1RAPL1, CPEB3, TNFRSF12A, HSPB1, OPRM1, LMX1A, CLCF1, ASPM, MECP2, NTF3, VEGFA, LRP2, FEZ1, ATP6VOC, RNASE6, CTSK, ACR, PRSS16, LAMP5, PRDX6, UNC13D, BAG3, TIAL1, ADRB2, HPS4, ASS1, CCKAR, GIMAP1-GIMAP5, HMOX1, SESN3, PCSK9, CAPN1, RNF152, VPS13C, DCN, and HMGB1.

In some embodiments, each of the one or more markers is a protein related to lysosomal activity. The protein related to lysosomal activity can be glycohydrolase or protease. The glycohydrolase can be selected from the group consisting of: β-hexosaminidase, β-galactosidase, β-galactosylcerebrosidase, β-glucuronidase. In some embodiments, the protease can be cathepsin. In some embodiments, the cathepsin is selected from the group consisting of: Cathepsin S, Cathepsin D, Cathepsin B, Cathepsin K, and Cathepsin L.

In some embodiments, the level of one or more markers previously known to be associated with a neurodegenerative disease are measured.

In some embodiments, the one or more markers are selected from the group consisting of (1) marker proteins previously known to be associated with a neurogenerative disease (e.g., AD); (2) a protein or mRNA encoded by a human homolog of the mouse gene selected from the group consisting of Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1; and (3) a protein related to lysosomal activity, such as glycohydrolase or protease. The glycohydrolase can be selected from the group consisting of: β-hexosaminidase, β-galactosidase, β-galactosylcerebrosidase, β-glucuronidase. In some embodiments, the protease can be cathepsin. In some embodiments, the cathepsin is selected from the group consisting of: Cathepsin S, Cathepsin D, Cathepsin B, Cathepsin K, and Cathepsin L.

In some embodiments, the level of the one or more markers is measured in a sample obtained after the step of commencing administration of trametinib. The sample can be obtained at one or multiple time points after the step of commencing administration of trametinib. For example, the sample is obtained 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks after commencing administration of trametinib. In some embodiments, the sample is obtained 1 month, 2 months, 3 months, 4 months, 5 months, or 6 months after commencing administration of trametinib. In some embodiments, the sample is obtained at once after the step of commencing administration of trametinib. In some embodiments, the sample is obtained at 2 different time points after the step of commencing administration of trametinib. In some embodiments, the sample is obtained at 3 different time points after commencing administration of trametinib. In some embodiments, the sample is obtained at 4, 5, 6, 7, or 8 different time points after the step of commencing administration of trametinib.

In some embodiments, the level of the one or more markers is measured in a control sample obtained before commencing administration of trametinib. In some embodiments, the level of one or more markers is measured in a biological sample obtained from healthy subjects who are free of the disease(s) of interest. In some embodiments, the method further comprises the step of comparing the level of one or more markers in the control sample obtained before commencing administration of trametinib and samples obtained after administration of trametinib. In some embodiments, the method further comprises the step of comparing the level of one or more markers in the samples from healthy subjects who are free of the disease(s) of interest to the level in the samples obtained from patients before commencing administration or after administration of trametinib. The comparison of the level of one or more marker proteins can be used to determine therapeutic effects of trametinib. In some embodiments, the level of one or more markers can be used to determine appropriate duration or dose of trametinib administration to achieve desired therapeutic outcome. In some embodiments, time-course analysis of the one or more markers is performed. In some embodiments, the level of one or more markers can be used to determine the methods of subsequent trametinib administration, such as duration and dose of trametinib. In some embodiments, the level of one or more markers can be used to identify individuals who are more likely than similar individuals without the biomarker to experience a favorable effect from exposure to trametinib.

The sample used for testing markers can be obtained by any of the methods known in the art. For example, the sample can be obtained by brain biopsy. In some embodiments, the sample is obtained by stereotactic brain biopsy. In some embodiments, the sample is obtained from body fluids or secretions of a patient, such as blood, cerebrospinal fluid (CSF), urine, body secreting fluid, saliva, stool, pleural fluid, lymphatic fluid, sputum, ascites, prostatic fluid, cerebrospinal fluid (CSF), or any other bodily secretion or derivative thereof. Blood sample includes whole blood, plasma, serum, peripheral blood mononuclear cells (PBMC), or any components of blood.

In another aspect, a composition for use in determining therapeutic effect of a MEK 1/2 inhibitor such as trametinib, comprising a probe for specifically detecting a marker is presented. In another aspect, kits for such purpose are also provided. Such kits may comprise a carrier being compartmentalized to receive in close confinement, one or more containers such as vials, tubes, and the like, each of the containers comprising one of the separate elements to be used in the method. For example, one of the containers may comprise a probe that is or can be detectably labeled. Such probe may be an antibody or polynucleotide specific for a protein or mRNA, respectively. Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application and may also indicate directions for either in vivo or in vitro use, such as those described above.

A typical embodiment is a kit comprising a container, a label on said container, and a composition contained within said container, wherein the composition includes a primary antibody that binds to a protein or autoantibody biomarker, and the label on said container indicates that the composition can be used to evaluate the presence of such proteins or antibodies in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label.

### 5.5. Pharmaceutical Compositions and Unit Dosage Form

In yet another aspect, the present disclosure provides a pharmaceutical composition and a unit dosage form comprising trametinib for treatment of a neurodegenerative disease (e.g., ALS).

In typical embodiments, trametinib is formulated for oral administration. In some embodiments, trametinib is formulation with an inert diluent or with an edible carrier. In various embodiments, trametinib is enclosed in hard or soft-shell gelatin capsules, compressed into tablets, or incorporated directly into the food of the diet. For oral therapeutic administration, the active compound may be incorporated with an excipient and used in the form of ingestible tablets, buccal tablets, coated tablets, troches, capsules, elixirs, dispersions, suspensions, solutions, syrups, wafers, patches, powder for oral solution and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for the material in a dosage form or pharmaceutical composition to be pharmaceutically pure and substantially non-toxic in the amounts employed.

Some compositions or dosage forms may be a liquid, or may comprise a solid phase dispersed in a liquid.

In some embodiments, an oral dosage form may comprise a silicified microcrystalline cellulose such as PROSOLV®. For example, about 20% (wt/wt) to about 70% (wt/wt), about 10% (wt/wt) to about 20% (wt/wt), about 20% (wt/wt) to about 40% (wt/wt), about 25% (wt/wt) to about 30% (wt/wt), about 40% (wt/wt) to about 50% (wt/wt), or about 45% (wt/wt) to about 50% (wt/wt) silicified microcrystalline cellulose may be present in an oral dosage form or a unit of an oral dosage form.

In some embodiments, an oral dosage form may comprise a crosslinked polyvinylpyrrolidone such as crospovidone. For example, about 1% (wt/wt) to about 10% (wt/wt), about 1% (wt/wt) to about 5% (wt/wt), or about 1% (wt/wt) to about 3% (wt/wt) crosslinked polyvinylpyrrolidone may be present in an oral dosage form or a unit of an oral dosage form.

In some embodiments, an oral dosage form may comprise a fumed silica such as AEROSIL®. For example, about 0.1% (wt/wt) to about 10% (wt/wt), about 0.1% (wt/wt) to about 1% (wt/wt), or about 0.4% (wt/wt) to about 0.6% (wt/wt) fumed silica may be present in an oral dosage form or a unit of an oral dosage form. In some embodiments, an oral dosage form may comprise magnesium stearate. For example, about 0.1% (wt/wt) to about 10% (wt/wt), about 0.1% (wt/wt) to about 1% (wt/wt), or about 0.4% (wt/wt) to about 0.6% (wt/wt) magnesium stearate may be present in an oral dosage form or a unit of an oral dosage form. An oral dosage form comprising zoledronic acid or another bisphosphonate may be included in a pharmaceutical product comprising more than one unit of the oral dosage form.

Trametinib may be formulated for other administration methods, for example, sublingual, rectal, intranasal, parenteral, transdermal or local administration, or injections. Solutions of the active compounds as free acids or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

In preferred embodiments, each unit of the oral dosage form contains an effective amount for daily administration. In some embodiments, each unit of the oral dosage form contains between 0.1 and 3 mg of trametinib. In some embodiments, each unit of the oral dosage form contains between 0.2 and 3 mg of trametinib. In some embodiments, each unit of the oral dosage form contains between 0.3 and 3 mg of trametinib. In some embodiments, each unit of the oral dosage form contains between 0.4 and 3 mg of trametinib. In some embodiments, each unit of the oral dosage form contains between 0.5 and 3 mg of trametinib. In some embodiments, each unit of the oral dosage form contains between 0.5 and 2.5 mg of trametinib. In some embodiments, each unit of the oral dosage form contains between 0.5 and 2 mg of trametinib. In some embodiments, each unit of the oral dosage form contains between 0.75 and 2.5 mg of trametinib. In some embodiments, each unit of the oral dosage form contains between 1 and 2 mg of trametinib. In some embodiments, each unit of the oral dosage form contains between 0.75 and 1.25 mg of trametinib. In some embodiments, each unit of the oral dosage form contains 0.2 mg of trametinib. In some embodiments, each unit of the oral dosage form contains 0.25 mg of trametinib. In some embodiments, each unit of the oral dosage form contains 0.5 mg of trametinib. In some embodiments, each unit of the oral dosage form contains 1 mg of trametinib. In some embodiments, each unit of the oral dosage form contains 1.5 mg of trametinib. In some embodiments, each unit of the oral dosage form contains 2 mg of trametinib. In some embodiments, each unit of the oral dosage form contains 2.5 mg of trametinib. In some embodiments, each unit of the oral dosage form contains 3 mg of trametinib.

In some embodiments, each unit of the oral dosage form is a MEKINIST tablet containing 0.5 mg, 1 mg, or 2 mg of trametinib. In some embodiments, each 0.5 mg tablet contains 0.5635 mg trametinib dimethyl sulfoxide equivalent to 0.5 mg of trametinib nonsolvated parent. In some embodiments, each 1 mg tablet contains 1.127 mg trametinib dimethyl sulfoxide equivalent to 1 mg of trametinib non-solvated parent. In some embodiments, each 2 mg tablet contains 2.254 mg trametinib dimethyl sulfoxide equivalent to 1 mg of trametinib non-solvated parent.

In some embodiments, the tablet contains from about 25% to about 89% by weight of one or more excipients. In some embodiments, the excipients are substantially free of water. The one or more excipients can be selected from the group consisting of microcrystalline cellulose, powdered cellulose, pregelatinized starch, starch, lactose, Di-calcium phosphate, lactitol, mannitol, sorbitol and maltodextrin. In some embodiments, the amount of unsolvated trametinib does not exceed about 20%. Pharmaceutical composition described in U.S. Pat. Nos. 8,580,304 and 9,271,941, incorporated by reference in their entireties, can be used for various embodiments of the present disclosure.

The tablet can further comprise a tablet core, containing colloidal silicon dioxide, croscarmellose sodium, hypromellose, magnesium stearate (vegetable source), mannitol, microcrystalline cellulose, and sodium lauryl sulfate. The tablet can further comprise a coating containing hypromellose, iron oxide red, iron oxide yellow, polyethylene glycol, polysorbate 80, and/or titanium dioxide.

In some embodiments, each unit of the oral dosage form is a disintegrating tablet containing between 0.1 and 3 mg of trametinib. In some embodiments, each unit of the oral dosage form is a disintegrating tablet containing between 0.2 and 2.5 mg of trametinib. In some embodiments, each unit of the oral dosage form is a disintegrating tablet containing between 0.3 and 2.25 mg of trametinib. In some embodiments, each unit of the oral dosage form is a disintegrating tablet containing between 0.5 and 2 mg of trametinib. In some embodiments, each unit of the oral dosage form is a disintegrating tablet containing 0.5 mg, 1 mg, or 2 mg of trametinib.

In some embodiments, each unit dosage form is powder containing between 0.1 and 3 mg of trametinib formulated for buccal or sublingual sprays. In some embodiments, each unit dosage form is powder containing between 0.2 and 2.5 mg of trametinib formulated for buccal or sublingual sprays. In some embodiments, each unit dosage form is powder containing between 0.3 and 2.25 mg of trametinib formulated for buccal or sublingual sprays. In some embodiments, each unit dosage form is powder containing between 0.5 and 2 mg of trametinib formulated for buccal or sublingual sprays. In some embodiments, each unit dosage form is powder containing 0.5 mg, 1 mg, or 2 mg of trametinib formulated for buccal or sublingual sprays.

### 5.6. EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art.

### 5.6.1. Example 1: Crossing of Blood-Brain Barrier

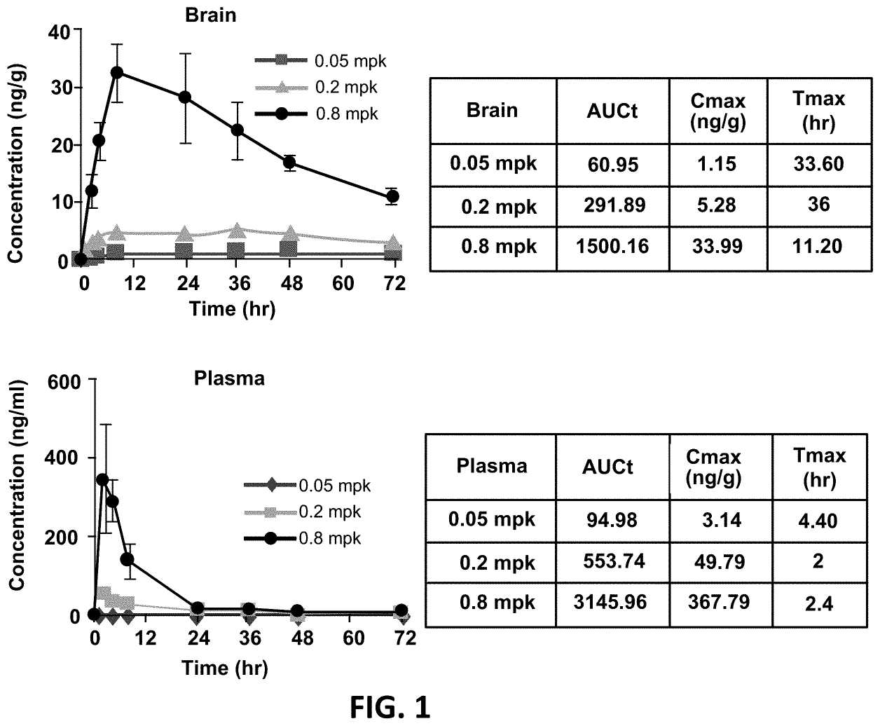

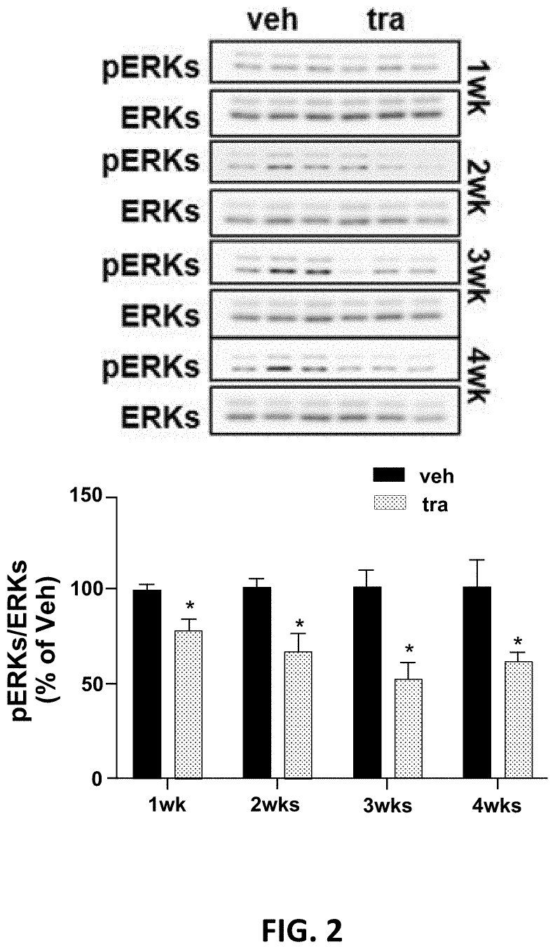

Trametinib was confirmed to penetrate the blood-brain barrier (BBB) after a single oral administration to wild type mice. The brain/plasma exposure ratio (AUC) was 47.7% in the highest dose group (FIG. 1). Trametinib was also found to exert its MEK1/2 inhibition in the brains of wild type mice after oral administration by causing significant decrease in pERK expression (FIG. 2). These results suggest that trametinib penetrates the BBB.

### 5.6.2. Example 2: Time-Course of Gene Expression Changes in the Brain after Administration of Trametinib

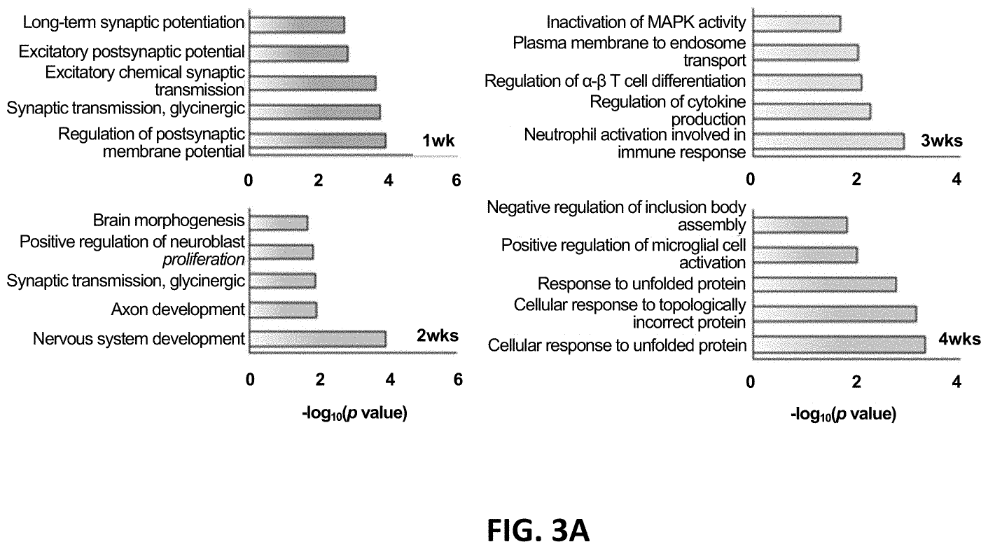

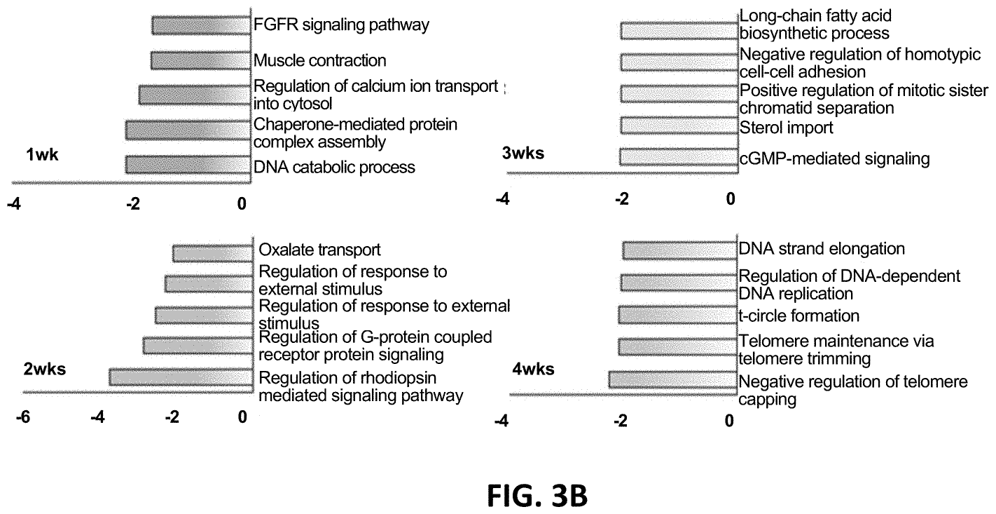

To evaluate the transcriptional profiles in the brain, bulk RNA-Seq was performed using whole brains of wild type mice following oral administration of trametinib. Gene ontology terms were enriched at each weekly time point, indicating the relevance of cellular function such as synaptic potential, nervous system development, immune response, and incorrect protein folding in a temporal pattern (FIG. 3A). Decrease in MEK-ERK signaling with trametinib administration during week 1 and week 2 of administration can be indirectly confirmed through the decrease in expression of FGF receptor signaling and GPCR signaling related genes. Decrease in expression of telomere related genes seen in week 4 of trametinib administration (FIG. 3B) suggests it is closely related to neuronal maturation or terminally differentiated neurons through the activation of neurogenesis.

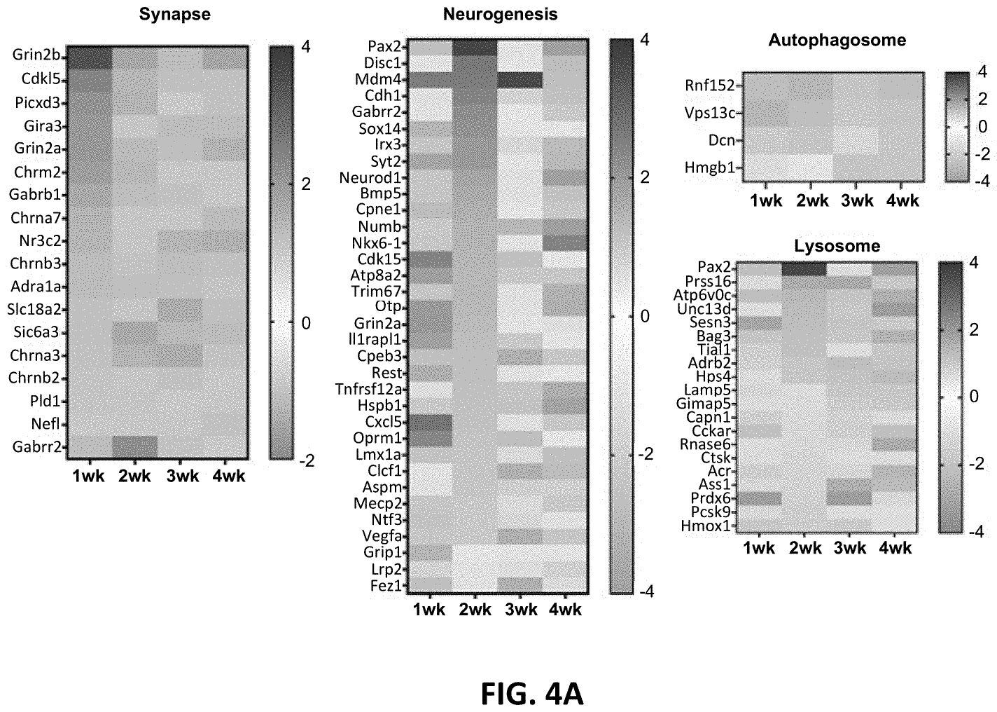

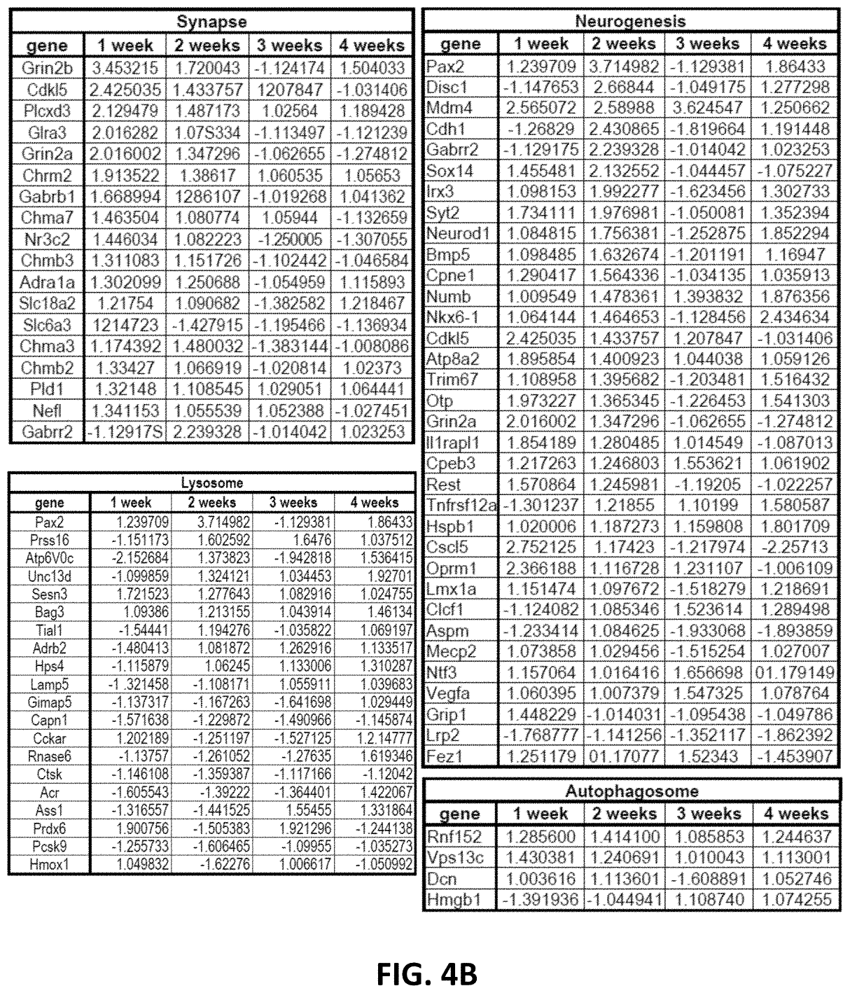

Our result demonstrates that the first week of trametinib administration appears to be the critical period for building neuronal communications, as evidenced by the transcriptional changes for synapse formation. In the second week, we observed neuroblast proliferation-related and axon growth-related gene expression, followed by expression of genes for immune reaction and incorrect protein modification reaction in the third and fourth week, respectively. It is important to note that increase in the gene-sets associated with neurogenesis and the induction of lysosomal activity both occurred within the four-week period, as illustrated by the gene expression heatmap (FIGS. 4A-4B). Genes set forth in FIG. 4A-4B are those that showed an absolute value of fold change (FC) of at least 1.3 between the vehicle-treated group and trametinib-treated group in at least one of the 1, 2, 3 or 4 week administration. Their FCs were between −2.26 and 3.71.

The proteins or mRNA encoded by a human homolog of the genes indicated in FIGS. 4A-4B or neurotransmitters relating to the protein receptors (GABA, glutamate, acetylcholine, monoamines such as dopamine, and the like) can be used as biomarkers for determining whether a beneficial effect has occurred in an individual who has been exposed to trametinib for the treatment of a neurodegenerative disease. The genes are Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1. The genes are related to synapse formation, neurogenesis, lysosomal function, and/or autophagosome.

### 5.6.3. Example 3: Therapeutic Effects of Trametinib in Rodents

Therapeutic efficacy of trametinib was examined in the SOD1-G93A transgenic mouse model, the most widely used animal model for ALS research. The SOD1-G93A ALS model mice, TgN [B6SJL-Tg (SOD1-G93A) 1Gur], were purchased from Jackson Laboratory (ME, USA) and bred at 21±2° C., 35-65% RH.

SOD1-G93A mice were orally treated with trametinib every day starting from the day when the mice showed ALS symptoms, after which they were subjected to behavioral tests such as grip strength and motor performance and checked for their survival time. In addition, immunohistochemical analysis was performed with the spinal cords of the mice to evaluate the efficacy of trametinib on the tissue, cellular, and molecular level.

In order to determine the timing of onset of ALS symptoms, the mice were subjected to a rotarod test with the LE8205 rotarod treadmill (Panlab/Harvard Apparatus, Spain). The timing of onset of ALS symptoms was defined as the day when, for two consecutive days, it takes fewer than 420 seconds for the animal on the rod rotating at 15 rpm to lose balance and fall to the floor due to hind leg tremor and weakness.

After the onset of symptoms (around 16 weeks after birth), trametinib was orally administered to two groups of ALS model mice every day at the doses of 0.1 mg/kg or 0.2 mg/kg (mpk). As controls, wild type (WT) mice and ALS model mice were treated with vehicle.

TABLE 1
Administration scheme

| | | | Dose | Number of |
|---|---|---|---|---|
| Groups | Drugs | Genotype | (mg/kg/day) | animals |
| WT | Vehicle | Non-carrier | — | 10 |
| Vehicle | Vehicle | SOD1 | — | 10 |
| 0.1 mpk | Trametinib | SOD1 | 0.1 | 10 |
| 0.2 mpk | Trametinib | SOD1 | 0.2 | 10 |

* Trametinib: Administered in the form of a suspension of micronized trametinib DMSO solvate in 5% mannitol + 1.5% HPMC + 0.2% SLS.

5.6.3.1. Grip Strength Test

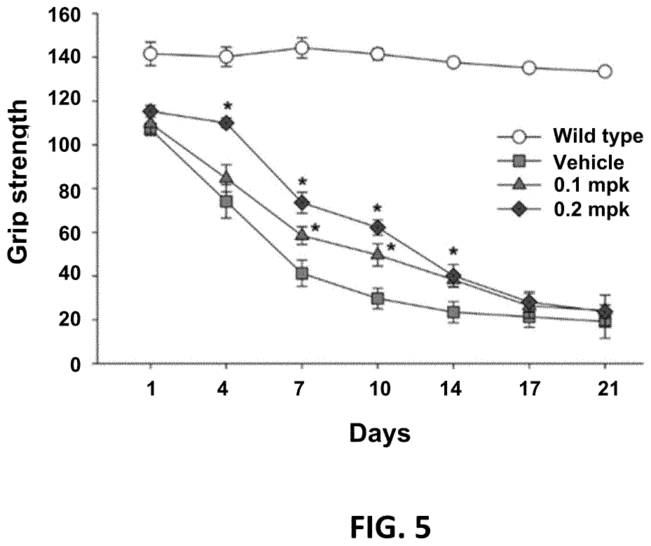

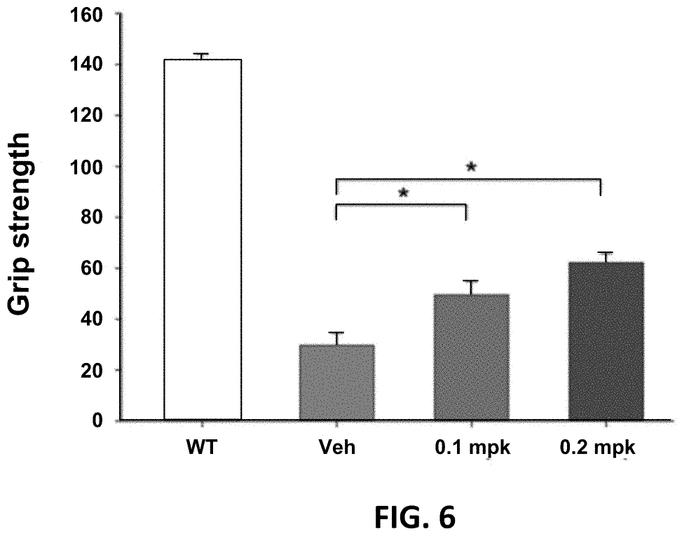

Grip strength test was performed with the Bioseb Grip Strength Test (BIO-G53). After the onset of ALS symptoms, the mice are maneuvered to grab the tester and then gently pulled away from the tester by the tail. The maximum force developed by the mouse was recorded. The 0.1 mg/kg/day group and the 0.2 mg/kg/day group showed significant difference in the grip strength at Days 7-10 and Days 4-14, respectively, compared to the vehicle treated group (FIGS. 5-6). These results show that trametinib delayed the rate of muscular strength decline in the ALS model mice for a certain time period.

5.6.3.2. Motor Performance Test

After the onset of ALS symptoms, motor performance was measured daily for each anatomic site based on the criteria as listed in the table below. The motor performance score was presented as the sum of the scores for each site.

TABLE 2
Motor performance score

| Site | Score | Criteria |
|---|---|---|
| Hind limbs | 3 | No weakness |
| | 2 | One leg paresis |
| | 1 | Paraparesis |
| | 0 | Paraplegia (no leg movement) |
| Fore limbs | 3 | No weakness |
| | 2 | paresis |
| | 1 | plegia |
| | 0 | Unable to lift up |
| Trunk | 1 | Able to twist |
| | 0 | Unable to twist |
| Total body motility | 3 | Able to run |
| | 2 | Able to walk or crawl |
| | 1 | Able to right itself |
| | 0 | Unable to right itself within 30 s |

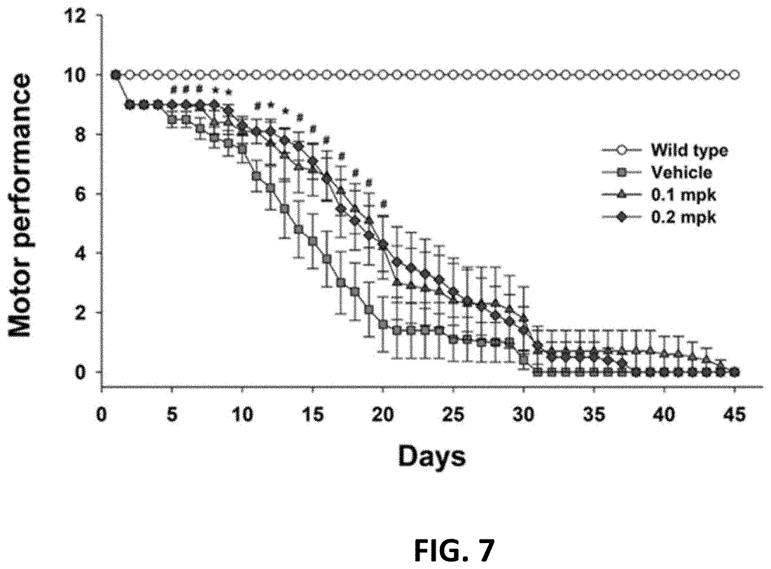

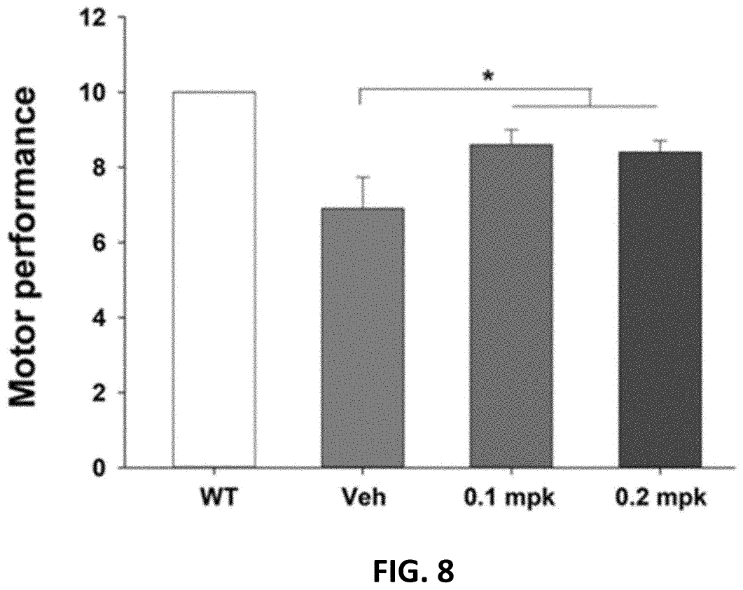

Trametinib-administered groups showed significant difference in the motor performance scores on Days 5 to 20 of administration compared to the vehicle-treated group (FIGS. 7-8). This result shows that trametinib reduces the rate of motor performance decline in the ALS model mice.

5.6.3.3. Survival

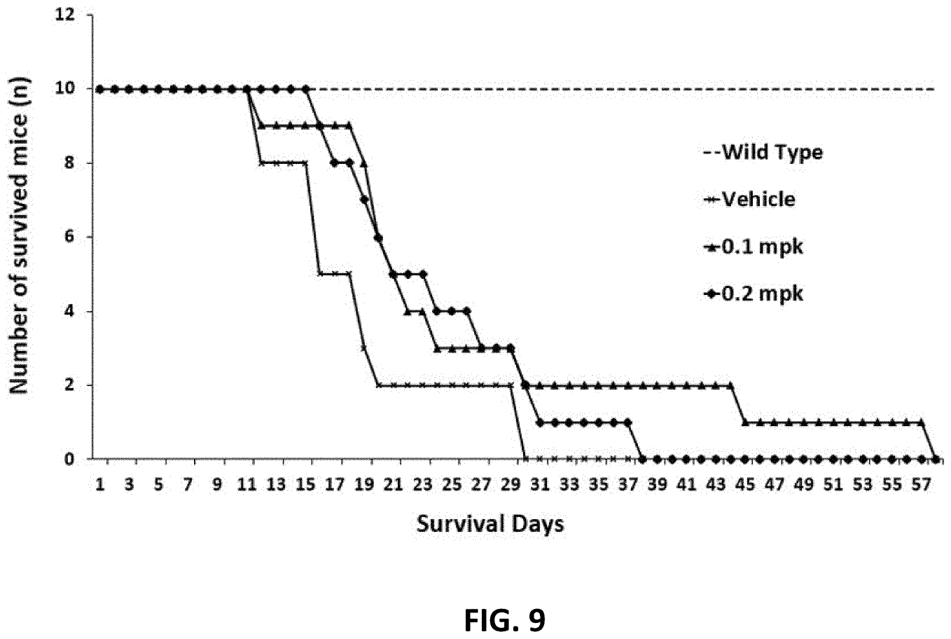

Mice with motor performance score at or lower than 3 were euthanized as they were not able to feed themselves. Survival days for each group of mice were counted. Survival was prolonged in trametinib-treated groups compared to the vehicle-treated groups as shown in FIG. 9.

5.6.3.4. Histological Analysis

After the behavioral tests above were completed, the spinal cords of the euthanized mice were removed, fixed, and processed. The spinal cord tissue was sliced to a thickness of 10 μm with the CM1860 cryomicrotome (Leica Biosystems, Germany). Immunohistochemical analysis was performed using various staining methods to examine the spinal cord tissue, motor neurons, and protein expression in the motor neurons.

Nissl Staining:

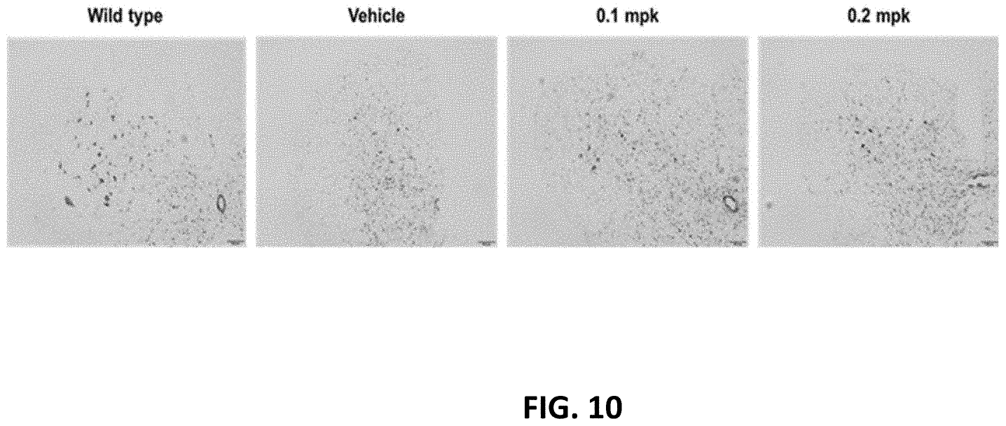

Through Nissl staining, the condition and distribution of neurons can be examined. Nissl staining solution contained 0.1% w/v Cresyl Violet (Sigma, C-5042, USA) and 0.1% v/v glacial acetic acid. Nissl staining of the spinal cord tissue revealed that the number of neurons (distinctly shaped cells stained in deep blue color in FIG. 10) decreased in the vehicle-treated ALS mice group compared to the wild type mice. In contrast, the trametinib-treated groups retained more neurons than the vehicle-treated group.

Motor Neurons:

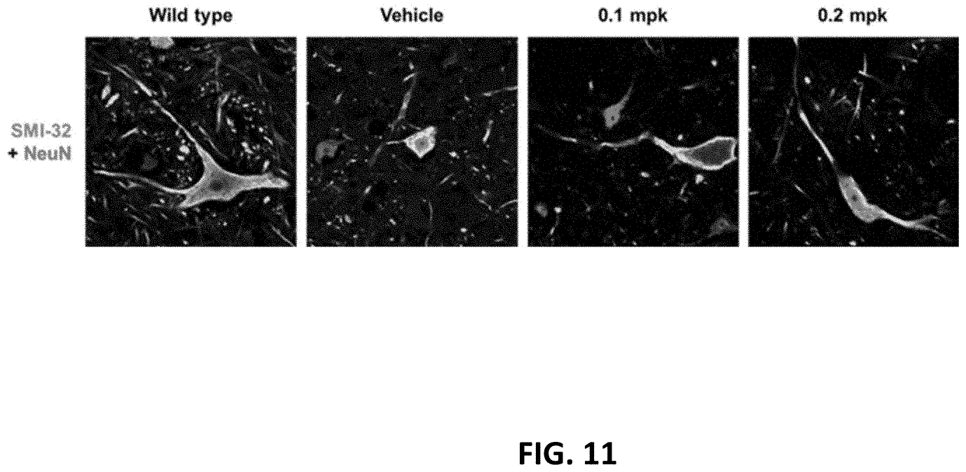

In order to examine the condition of the motor neurons, the spinal cord tissues were subjected to NeuN immunofluorescent staining to observe all types of neurons (red in FIG. 11) and SMI-32 staining (SMI-32 antibody binds specifically to motor neurons) to observe motor neurons (green in FIG. 11). As can be seen from FIG. 11, the vehicle-treated group showed a decrease in the number of neurons and motor neurons with severe damage in the shape of the motor neurons (small-sized cell bodies and lack of elongated neurites). In contrast, the elongated neurite shape was relatively well preserved in the trametinib-treated groups compared to the vehicle-treated group.

Axonal Tract:

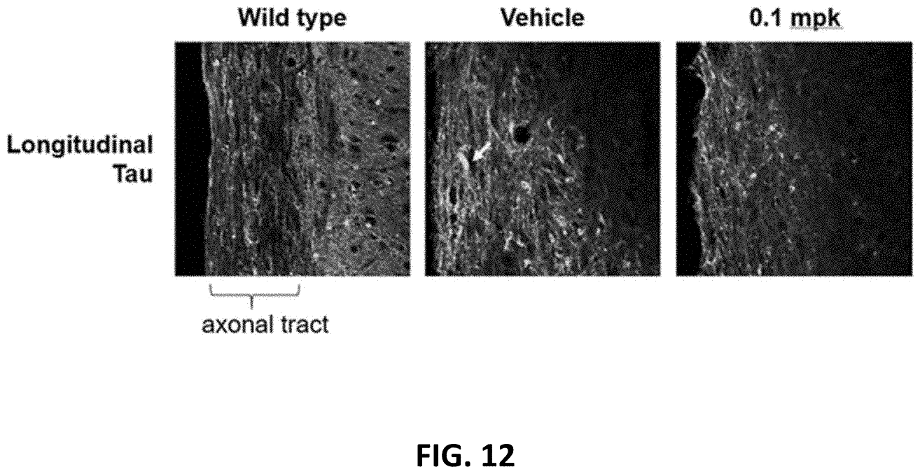

A longitudinal section of the spinal cord tissue was stained with tau antibody to examine the condition of the axonal nerve tract. While long and straight axon tracts were clearly observed in wild type mice, numerous axonal swelling (yellow arrow in FIG. 12) and spheroids were observed in the vehicle-treated mice. In the trametinib-treated mice, a reduced number of abnormal axonal spheroids was seen compared to the vehicle-treated group (FIG. 12).

Axon:

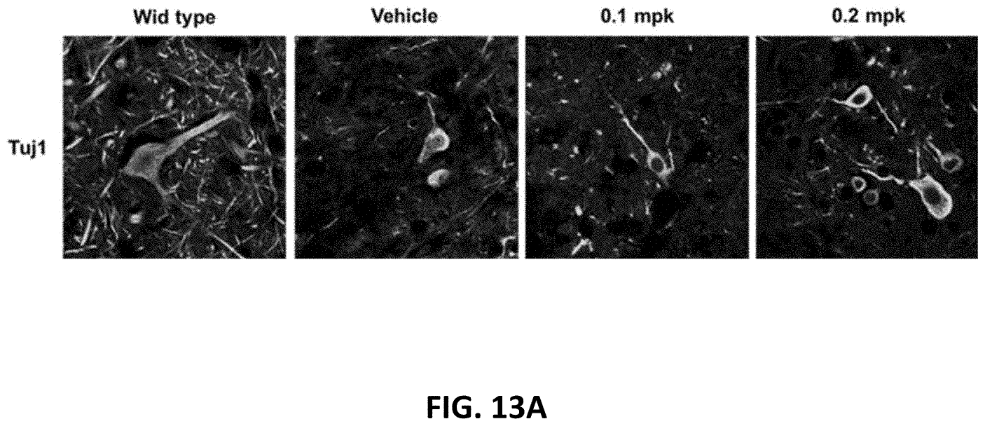

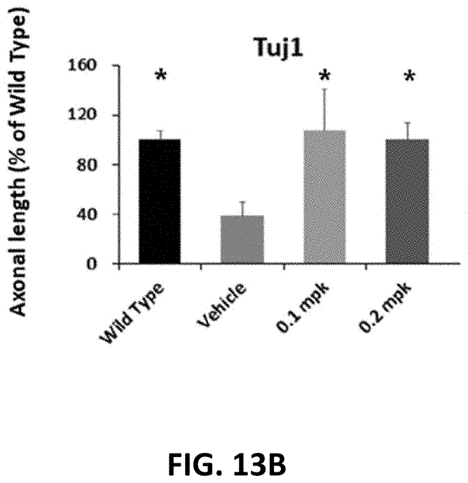

A cross-section of the spinal cord was stained with Tuj 1, an axonal marker, to examine the condition of axons in the tested mice. Trametinib-treated mice showed relatively well protected long axons projecting out of the cell body compared to the vehicle-treated ALS mice (FIGS. 13A and 13B).

Dendrite:

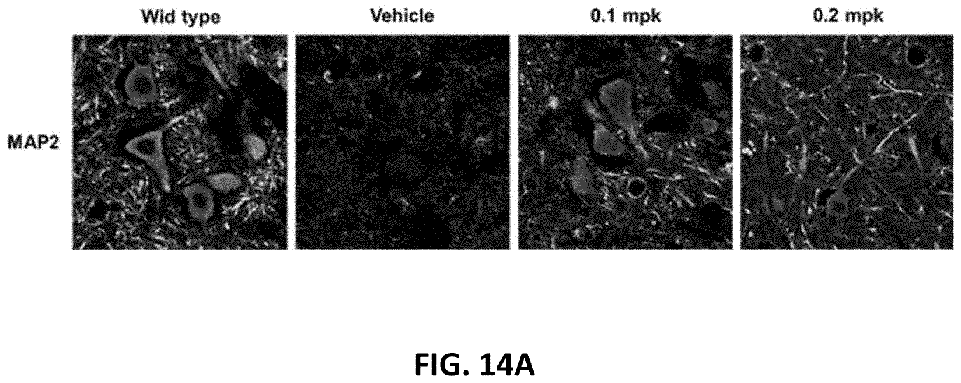

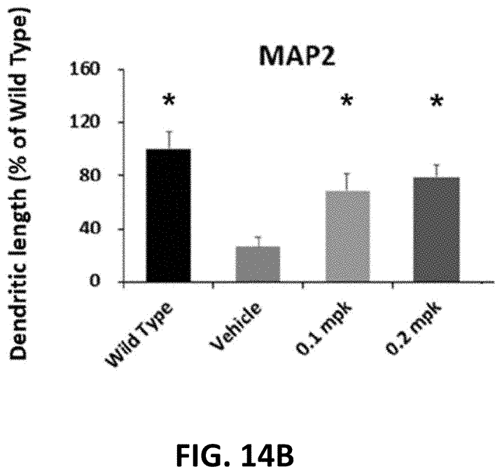

MAP2 staining was performed to observe the condition of dendrites projecting out of the neurons. In vehicle-treated ALS mice, dendrites were severely damaged to the extent that stained MAP2 was hardly observed. In contrast, the dendrites in the trametinib-treated groups were quite well protected compared to the vehicle-treated mice (FIGS. 14A and 14B).

### 5.6.4. Example 4: Enhancement of Lysosomal Activity by Trametinib Through Autophagosome-Lysosome Fusion

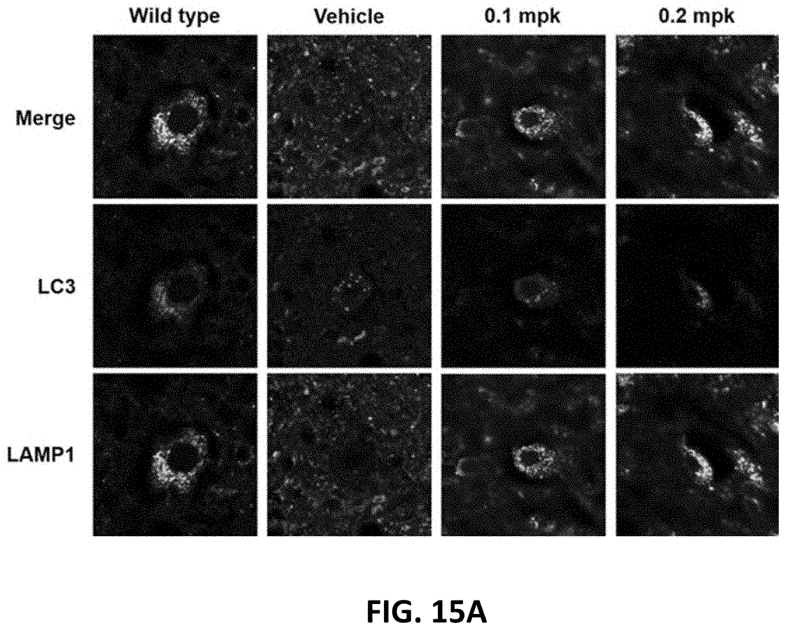

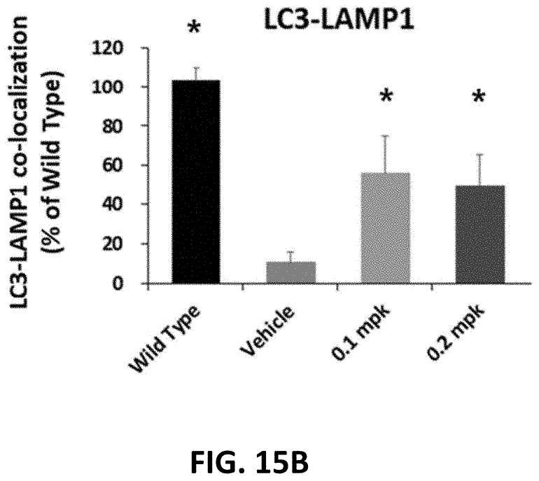

The expression of LC3, an autophagosome marker (red fluorescence in the second row of FIG. 15A), and LAMP1, a lysosome marker (green fluorescence in the third row of FIG. 15A), was examined in the spinal cord tissues using immunofluorescence staining. Vehicle-treated ALS model mice showed a significant decrease in LAMP1 signal compared to the wild type mice (green fluorescence in the third row of FIG. 15A). In the trametinib-treated mice, LAMP1 signal was restored compared to the vehicle-treated ALS model mice, and the fusion of lysosome and autophagosome (merged yellow fluorescence in the first row FIG. 15A) was restored as well (FIGS. 15A and B).

These results suggest that endogenous molecules related to lysosomal activity can be used as a biomarker for determining whether trametinib has caused a beneficial effect in an individual suffering from ALS. Such molecules include glycohydrolases such as β-hexosaminidase, β-galactosidase, β-galactosylcerebrosidase, β-glucuronidase and proteases such as cathepsins including Cathepsin S, Cathepsin D, Cathepsin B, Cathepsin K, Cathepsin L.

In this study, we demonstrated that trametinib rescues autophagic flux and lysosomal activity in the pathologic environment via induction of autophagosome-lysosome fusion. The maintenance of proteostasis through lysosomal activation not only induces protection through neurotoxin removal, but also reverses age-related phenotype (rejuvenation) through intracellular metabolic activation. Accordingly, the induction of lysosomal activation and neurogenesis possibly act synergistically to bring about the recovery effect in the ALS patients.

### 5.6.5. Example 5: Therapeutic Effects of Trametinib in Humans

Patients are selected based on the following criteria for a sequential dose-escalation, randomized, active-controlled, phase ½a clinical trial to evaluate the safety, tolerability and efficacy of trametinib in patients with ALS.

Inclusion Criteria:

Patients diagnosed as definite, probable or probable-laboratory-supported ALS according to El Escorial Criteria. Patients of less than 2 years after the onset of ALS. Patients who meet the criteria of K-ALSFRS-R score (total score of at least 30 and score of at least 2 for each item) and forced vital capacity (% FVC≥70%, or 60%≤% FVC<70% with K-ALSFRS-R score ≥3 for items 10 to 12).

Exclusion Criteria:

Patients with primary lateral sclerosis, progressive muscular atrophy or lower motor neuron disease. Patients who have history of ALS treatment of edaravone or stem cell therapy within 16 weeks before screening. Patients who have permanently ceased the administration of riluzole due to lack of tolerability and/or efficacy. Patients in Class II to IV according to the New York Heart Association functional classification. Patients with myocardial infarction, unstable arrhythmia, and/or significant cardiovascular disease such as unstable angina within 12 weeks before screening. Patients who do not meet the criteria of laboratory tests and medical/operation history.

Selected patients are randomly assigned 4:1 to receive trametinib or riluzole as an active comparator. The first group of patients receive trametinib (0.5 mg, 8 patients) or riluzole (100 mg, 2 patients). The initiation of the next dose groups (1 mg or 2 mg of trametinib) is decided by the IDMC (Independent Data Monitoring Committee) through safety assessment of the first 4-week periods of the first group.

As primary outcome measures, safety and tolerability of trametinib is tested based on observation of adverse events. As secondary outcome measures, K-ALSFRS-R (Korean version of Amyotrophic Lateral Sclerosis Functional Rating Scale-Revised) scores are measured in the patients by independent outcome assessors. Additionally, change in forced vital capacity (FVC) from baseline, trough concentrations of trametinib in cerebrospinal fluid (CSF) and trough concentrations of trametinib in plasma are further measured.

Patients treated with trametinib show reduction of a behavioral, physiological and/or pathological symptom associated with ALS.

Some patients further have change in the level of one or markers after treatment with trametinib. The one or more markers are (1) proteins encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1; and (2) proteins related to lysosomal activity, particularly cathepsin including Cathepsin S, Cathepsin D, Cathepsin B, Cathepsin K, and Cathepsin L.

### 5.6.6. Experimental Methods

Trametinib Treatment:

Trametinib (Medchemexpress, Monmouth Junction, N.J.) was micronized and suspended in the vehicle containing 5% mannitol, 1.5% hydroxypropyl methylcellulose and 0.2% sodium lauryl sulfate. For pharmacokinetic analysis, 0.05, 0.2 and 0.8 mg/kg of trametinib were orally administered in to 7-week-old normal mice (n=5 per group) as a single administration. Mice were sacrificed at each identical time points. SOD1-G93A mice (female, n=10 per group) were orally treated with trametinib every day starting from the day when the mice showed ALS symptoms. All the mice were sacrificed by the perfusion method and brain or spinal cord samples were processed for biochemical and immunohistochemical analysis.

Whole Cell RNA Sequencing:

RNA was isolated from the mice whole brain and cDNA libraries for RNA sequencing were prepared using the TruSeq Stranded mRNA Prep Kit (Illumina, San Diego, Calif.) according to the manufacturer's guidelines (l). The libraries were sequenced on the Illumina Nextseq500 platform and the reads were mapped to the reference Mouse mm10 genome using Tophat v2.0.13. The total number of reads mapped to the transcriptomes were 24,532 genes and the genes with 0 count in at least one sample were removed before differential expression analysis. There was a total of 18,727 genes after the removal of genes with 0 count. To define differentially expressed genes (DEG), we set up a stringent statistic cutoff of fold change (FC) of ≥1.3 and a false discovery rate (FDR)<0.05. A total of 500 DEGs was identified between the vehicle-treated group and trametinib-treated group in the first week, 498 DEGs in the second week, 446 genes in the third week and 538 genes in the fourth week. Gene ontology was performed with Gene Ontology program of the gene ontology consortium. Heatmap analysis was performed by R studio using the DEG list related with synapse, neurogenesis and lysosome.

Immunohistochemical Analysis:

Mice were perfused with ice-cold phosphate buffered saline (PBS) and followed by 4% paraformaldehyde (PFA). Spinal cords were dissected and analyzed by immunohistochemistry. For cryosections, spinal cords were prepared into sections of 10 μm slices. For immunostaining, the sections were incubated with anti-Map2 (Millipore, Burlington, Mass.), anti-Tuj 1 (Cell signaling, Danvers, Mass.), anti-SMI-32 (Biolegend, San Diego, Calif.), anti-LAMP1 (Abcam, Cambridge, UK), or anti-LC3 (Cell signaling) antibodies. This step was followed by incubation with Alexa Fluor 488-conjugated anti-mouse IgG (Thermo, Waltham, Mass.), Alexa Fluor 488-conjugated anti-rat IgG (Thermo), Alexa Fluor 555-conjugated anti-rabbit IgG secondary antibodies (Thermo). The sections were counterstained with DAPI. The immunofluorescent images were captured using a LSM700 Laser-Scanning confocal microscope (Carl Zeiss, Heildenheim, Germany). For Nissl staining, the sections were incubated with Nissl staining solution (0.1% w/v Cresyl Violet (Sigma, C-5042, USA) and 0.1% v/v glacial acetic acid). The length of the Map2 and Tuj 1 positive dendrites and axons were measured using Icy program. The LC3-LAMP1 co-localization was observed under a confocal microscopy using a LSM700 microscope (Carl Zeiss).

Protein Extraction and Western Blotting:

Cells and tissues were washed twice with ice-cold phosphate-buffered saline (PBS) and extracted by homogenizing with Ripa buffer (10 mM HEPES, 1.5 mM MgCl₂, 10 mM KCl, 0.01 M DTT, protease inhibitors, pH 7.9). Lysates were centrifuged at 13,000 rpm for 20 min at 4° C. and the protein content in the supernatant was determined using Bradford assay (Bio-rad, Hercules, Calif.). Protein from each sample was subjected to 8%-15% SDS-PAGE, and the resolved proteins were transferred to nitrocellulose or polyvinylidene fluoride membrane. The membranes were blocked with 5% nonfat milk powder in Tris-buffered saline/Tween 20 (TBST) for 1 h at room temperature, then incubated with anti-phospho-ERK (Cell signaling), anti-ERK (Cell signaling), anti-LAMP1 (Abcam), anti-LC3 (Cell signaling), anti-cathepsin B (Cell signaling), anti-p62 (Cell signaling), anti-p62 (5114, Cell signaling), anti-phospho-mTOR (5536, Cell signaling), anti-mTOR (2983, Cell signaling), anti-phospho-ULK1 (14202, Cell signaling), anti-ULK1 (8054, Cell signaling), anti-TFEB (852501, Biolegend), and anti-GAPDH (Cell signaling) overnight at 4° C. After washing, membranes were incubated with horseradish peroxidase-conjugated goat anti-rabbit IgG antibody (Thermo), or goat anti-rat IgG antibody (Thermo) for 2 h at room temperature. Peroxidase activity was visualized with enhanced chemiluminescence. The detected signals were quantified using a LAS-4000 system (Fuji Film, Tokyo, Japan).

### 6. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 7. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A method of treating amyotrophic lateral sclerosis (ALS), comprising the step of administering an effective amount of trametinib to a patient diagnosed with ALS.

2. The method of claim 1, wherein trametinib is administered at an oral dose between 0.5 and 2 mg/day.

3. The method of claim 1, wherein trametinib is administered at an oral dose of 0.5 mg/day.

4. The method of claim 1, wherein trametinib is administered at an oral dose of 1 mg/day.

5. The method of claim 1, wherein trametinib is administered at an oral dose of 2 mg/day.

6. The method of claim 1, wherein trametinib is administered at a dose that provides a mean peak trametinib concentration (Cₘₐₓ) of at least 0.25 ng/g in the brain.

7. The method of claim 6, wherein trametinib is administered at a dose that provides a mean peak brain trametinib concentration (Cₘₐₓ) of at least 0.5 ng/g, at least 0.75 ng/g, at least 1 ng/g, at least 1.5 ng/g, at least 2 ng/g, at least 5 ng/g, or at least 10 ng/g in the brain.

8. The method of claim 6, wherein trametinib is administered at a dose that provides a mean peak brain trametinib concentration (Cₘₐₓ) of between 0.25 and 20 ng/g in the brain.

9. The method of claim 1, wherein trametinib is administered at a dose that provides a mean peak trametinib concentration (Cₘₐₓ) of at least 2 ng/ml in the plasma.

10. The method of claim 9, wherein trametinib is administered at a dose that provides a mean peak trametinib concentration (Cₘₐₓ) of at least 3 ng/ml, at least 10 ng/ml, at least 20 ng/ml, at least 30 ng/ml or at least 40 ng/ml in the plasma.

11. The method of claim 9, wherein trametinib is administered at a dose that provides a mean peak trametinib concentration (Cₘₐₓ) of between 2 and 100 ng/ml in the plasma.

12. The method of claim 1, wherein trametinib is administered at a daily oral dose effective to induce change in the level of one or more markers in the patient's brain or in a biological sample obtained from the patient of at least 1.3 fold after at least four weeks' administration as compared to prior to administration of trametinib.

13. The method of claim 12, wherein the daily oral dose is effective to induce change in the level of the one or more markers in the patient's brain or in the biological sample of at least 1.5 fold, at least 2 fold, at least 3 fold, at least 4 fold, at least 5 fold, at least 6 fold, at least 7 fold, at least 8 fold, at least 9 fold, at least 10 fold, at least 20 fold, at least 50 fold, or at least 100 fold after at least four weeks' administration as compared to prior to administration of trametinib.

14. The method of claim 1, wherein trametinib is administered at a daily oral dose effective to decrease the level of one or more markers in the patient's brain or in a biological sample obtained from the patient by at least 20% after at least four weeks' administration as compared to prior to administration of trametinib.

15. The method of claim 14, wherein the daily oral dose is effective to decrease the level of the one or more markers in the patient's brain or in the biological sample by at least 30%, at least 40%, at least 50%, at least 60%, at least 70%, at least 80%, at least 90%, or at least 99% after at least four weeks' administration as compared to prior to administration of trametinib.

16. The method of claim 12, wherein each of the one or more markers is encoded by a human homolog of the mouse gene selected from the group consisting of: Gabrb1, Gabrr2, Glra3, Nr3c2, Cdkl5, Grin2a, Grin2b, Plcxd3, Chrm2, Chrna3, Chrna7, Chrnb2, Nef1, Pld1, Adra1a, Chrnb3, Slc6a3, Slc18a2, Cdh1, Neurod1, Nkx6-1, Cxcl5, Rest, Syt2, Disc1, Irx3, Mdm4, Sox14, Grip1, Pax2, Bmp5, Cpne1, Numb, Atp8a2, Trim67, Otp, Illrapl1, Cpeb3, Tnftsfl2a, Hspb1, Oprm1, Lmx1a, Clcf1, Aspm, Mecp2, Ntf3, Vegfa, Lrp2, Fez1, Atp6vOc, Rnase6, Ctsk, Acr, Prss16, Lamp5, Prdx6, Uncl3d, Bag3, Tial1, Adrb2, Hps4, Ass1, Cckar, Gimap5, Hmox1, Sesn3, Pcsk9, Capn1, Rnf152, Vps13c, Dcn, and Hmgb1.

17. The method of claim 12, wherein each of the one or more markers is a protein related to lysosomal activity.

18. The method of claim 12, wherein each of the one or more markers is a cathepsin.

19. The method of claim 19, wherein the cathepsin is selected from the group consisting of: Cathepsin S, Cathepsin D, Cathepsin B, Cathepsin K, and Cathepsin L.

20. The method of claim 1, further comprising the step of measuring the level of one or more markers in a sample obtained from the patient.

21. The method of claim 20, wherein the sample is obtained after the step of administering trametinib.

22. The method of claim 20, further comprising the step of measuring the level of the one or more markers in a control sample obtained from the patient before the step of administering trametinib.

23. The method of claim 20, wherein the sample is obtained by brain biopsy, or from blood or cerebrospinal fluid (CSF) of the patient.

24. The method of claim 20, further comprising the step of determining therapeutic efficacy of trametinib administered to the patient based on the level of the one or more markers.

25. The method of claim 20, further comprising the step of comparing the level of the one or more markers in a sample obtained from the patient to the level of the one or more markers in samples from healthy subjects.

26. The method of claim 24, further comprising the step of discontinuing administration of trametinib based on determination of the therapeutic efficacy.

27. The method of claim 24, further comprising the step of continuing administration of trametinib based on determination of the therapeutic efficacy.

28. The method of claim 24, further comprising the step of adjusting administration of trametinib based on determination of the therapeutic efficacy.
